(19) United States
(12) Patent Application Publication
(10) Pub. No.: US 2009/0283723 A1
(43) Pub. Date: Nov. 19, 2009

(54) OLIGOMERIC COMPOUNDS WHICH FORM A SEMICONDUCTOR LAYER

(75) Inventors: Timo Meyer-Friedrichsen, Krefeld (DE); Stephan Kirchmeyer, Leverkusen (DE); Andreas Elschner, Mulheim (DE); Sergei Anatolievich Ponomarenko, Moskau (RU)

Correspondence Address: CONNOLLY BOVE LODGE & HUTZ, LLP, P O BOX 2207, WILMINGTON, DE, 19899, US

(73) Assignee: H.C. Starck Gmbh, Goslar (DE)

(21) Appl. No.: 12/441,153

(22) PCT Filed: Sep. 11, 2007
(86) PCT No.: PCT/EP2007/059506

§ 371 Date: Mar. 13, 2009

(30) Foreign Application Priority Data
Sep. 14, 2006 (DE) 10 2006 043039.5

(51) Int. Cl. H01B 1/12; C07D 333/06

(52) U.S. Cl. 252/500; 549/4

(57) ABSTRACT

Oligomeric compounds of the general formula (M), mixtures thereof and electronic components containing the same:

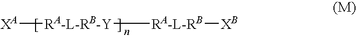

wherein L represents a linear conjugated oligomeric chain; wherein each R^{A} and each R^{B} independently represents a moiety selected from the group consisting of linear or branched C₂-C₂₀-alkylene radicals, C₃-C₈-cycloalkylene radicals, mono- or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals; wherein X^{A} and X^{B} each independently represents a moiety selected from optionally substituted vinyl groups, chlorine, iodine, hydroxyl, alkoxy groups having 1 to 3 carbon atoms, alkoxysilyl groups, silyl groups, chlorosilyl groups, siloxane groups, carboxyl groups, methyl or ethyl carbonate groups, aldehyde groups, methylcarbonyl groups, amino groups, amido groups, sulphone groups, sulphonic acid groups, halosulphonyl groups, sulphonate groups, phosphonic acid groups, phosphonate groups, trichloromethyl, tribromomethyl, cyanate groups, isocyanate groups, thiocyanate groups, isothiocyanate groups, cyano groups, nitro groups and H; wherein each Y independently represents a moiety selected from an optionally substituted alkoxysilylene group, silylene group, chlorosilylene group, methylene group, dichloromethylene group, dibromomethylene group, divalent siloxane group, disiloxane group, carboxyl group, carbonate group, amino group, amido group, sulphate group, phosphonate group, phosphate group, borate group, S and O; and wherein n represents an integer of 1 to 10.

The invention relates to oligomeric organic compounds and to mixtures thereof with macromolecular compounds having a core-shell structure and/or monomeric linear compounds, which have improved semiconductive properties, and to their use in electronic components.

The field of molecular electronics has developed rapidly in the last 15 years with the discovery of organic conductive and semiconductive compounds. In this time, a multitude of compounds which have semiconductive or electrooptical properties have been found. It is generally understood that molecular electronics will not displace conventional semiconductor units based on silicon. Instead, it is assumed that molecular electronic components will open up new fields of application in which suitability for coating large surfaces, structural flexibility, processibility at low temperatures and low costs are required. Semiconductive organic compounds are currently being developed for fields of use such as organic field-effect transistors (OFETs), organic luminescent diodes (OLEDs), sensors and photovoltaic elements. As a result of simple structuring and integration of OFETs into integrated organic semiconductor circuits, inexpensive solutions for smart cards or price tags, which have not been realizable to date with the aid of silicon technology owing to the cost and the lack of flexibility of the silicon units, are becoming possible. It would likewise be possible to use OFETs as switching elements in large-area flexible matrix displays. A review of organic semiconductors, integrated semiconductor circuits and their uses is given, for example, in Electronics 2002, Volume 15, p. 38 or H. Klauk (editor), Organic Electronics Materials, Manufacturing and Applications, Wiley-VCH 2006.

A field-effect transistor is a three-electrode element in which the conductivity of a thin conduction channel between two electrodes (known as “source” and “drain”) is controlled by means of a third electrode separated from the conduction channel by a thin insulator layer (known as “gate”). The most important characteristic properties of a field-effect transistor are the mobility of the charge carriers, which crucially determine the switching speed of the transistor and the ratio between the currents in the switched and unswitched state, the so-called “on/off ratio”.

In organic field-effect transistors, two major classes of compounds have been used to date. Compounds of the two classes have continuous conjugated units and are divided into conjugated polymers and conjugated oligomers according to molecular weight and structure.

Oligomers generally have a homogeneous molecular structure and a molecular weight below 10 000 daltons. Polymers consist generally of chains of homogeneous repeat units with a molecular weight distribution. However, there is a fluid transition between oligomers and polymers.

Frequently, the distinction between oligomers and polymers expresses the fact that there is a fundamental difference in the processing of these compounds. Oligomers are frequently evaporable and are applied to substrates by means of vapour deposition processes. Irrespective of their molecular structure, polymers frequently refer to compounds which are no longer evaporable and are therefore applied by means of other processes. In the case of polymers, compounds which are soluble in a liquid medium, for example organic solvents, are generally pursued, and can then be applied by means of appropriate application processes. A very widespread application method is, for example, the spin-coating method. A particularly elegant method is the application of semiconductive compounds via the inkjet method. In this method, a solution of the semiconductive compounds is applied to the substrate in the form of ultrafine droplets and dried. During the application, this method allows structuring to be performed. One description of this application process for semiconductive compounds is given, for example, in Nature, Volume 401, p. 685.

In general, a greater potential for arriving at inexpensive organic integrated semiconductor circuits in a simple manner is ascribed to the wet-chemical method.

An important prerequisite for the production of high-value organic semiconductor circuits is compounds of extremely high purity. In semiconductors, order phenomena play an important role. Hindrance of uniform alignment of the compounds and shapes of particle interfaces lead to a dramatic decline in the semiconductor properties, such that organic semiconductor circuits which have been formed using compounds not of extremely high purity are generally unusable. Remaining impurities can, for example, inject charges into the semiconductive compound (“doping”) and hence reduce the on/off ratio or serve as charge traps and hence drastically lower the mobility. Moreover, impurities can initiate the reaction of the semiconductive compounds with oxygen, and oxidizing impurities can oxidize the semiconductive compounds and hence shorten possible storage, processing and operating times.

The purity generally needed is so high that it is generally not achievable by the known polymer chemistry methods such as washing, reprecipitation and extraction. Oligomers, in contrast, as molecularly homogeneous and frequently volatile compounds, can be purified relatively simply by sublimation or chromatography.

Some important representatives of semiconductive polymers are described below. For polyfluorenes and fluorene copolymers, for example poly(9,9-dioctyl-fluorene-co-bithiophene) (I)

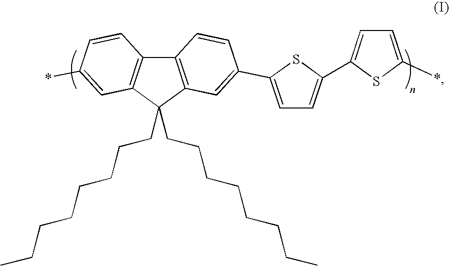

charge mobilities, also referred to hereinafter as mobilities for short, up to 0.02 cm²/Vs have been attained (Science, 2000, Volume 290, p. 2123), and, with regioregular poly(3-hexylthiophene-2,5-diyl) (II)

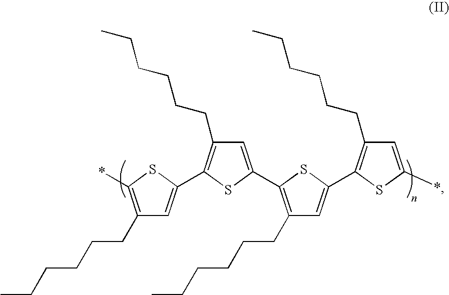

even mobilities up to 0.1 cm²/Vs (Science, 1998, Volume 280, p. 1741). Polyfluorene, polyfluorene copolymers and poly(3-hexylthiophene-2,5-diyl), like almost all long-chain polymers, form good films after application from solution and are therefore simple to process. As high molecular polymers with a molecular weight distribution, however, they cannot be purified by vacuum sublimation and can be purified only with difficulty by chromatography.

Important representatives of oligomeric semiconductive compounds are, for example, oligothiophenes, especially those with terminal alkyl substituents of the formula (III)

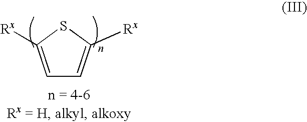

and pentacene (IV)

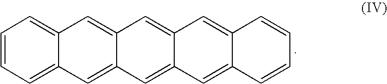

Typical mobilities, for example for α,α′-dihexyl-quarter-, -quinque- and sexithiophene, are 0.05-0.1 cm²/Vs. Oligothiophenes are generally hole semiconductors, i.e. exclusively positive charge carriers are transported.

The highest mobilities of a compound are obtained in single crystals; for example, a mobility of 1.1 cm²/Vs has been described for single crystals of α,α′-sexithiophene (Science, 2000, Volume 290, p. 963), and 4.6 cm²/Vs for single rubrene crystals (Adv. Mater., 2006, Volume 18, p. 2320). When oligomers are applied from solution, the mobilities usually fall significantly. In general, the fall in the semiconductive properties on processing of oligomeric compounds from solution is attributed to the moderate solubility and low tendency to form films of the oligomeric compounds. For instance, inhomogeneities are attributed, for example, to precipitations during the drying from the solution (Chem. Mater., 1998, Volume 10, p. 633).

There have therefore been attempts to combine the good processing and film formation properties of semiconductive polymers with the properties of semiconductive oligomers. The patent U.S. Pat. No. 6,025,462 describes conductive polymers with a star structure, which consist of a branched core and a shell composed of conjugated side groups. However, these have some disadvantages. When the side groups are formed from laterally unsubstituted conjugated structures, the resulting compounds are sparingly soluble or insoluble and not processible. When the conjugated units are substituted by side groups, this does lead to an improved solubility, but the side groups, as a result of their steric demands, bring about internal disorder and morphological disruption, which impair the semiconductive properties of these compounds.

The published specification WO 02/26859 A1 describes polymers composed of a conjugated backbone to which aromatic conjugated chains are joined. The polymers bear diarylamine side groups which enable electron conduction. However, these compounds are unsuitable as semiconductors owing to the diarylamine side groups.

The published specifications EP-A 1 398 341 and EP-A 1 580 217 describe semiconductive compounds having a core-shell structure which are used as semiconductors in electronic components and can be processed from solution. However, these compounds tend to give rise to poorly crystallizing films during processing, which, since crystallized films are a prerequisite for high charge carrier mobility, can be troublesome for some applications. Although it is known that films of organic semiconductors can be restructured by thermal treatment (deLeeuw et al. WO 2005104265), the macromolecular character of the compound can also hinder complete subsequent structuring by thermal treatment.

In Applied Physics Letters 90, 053504 (2007), Yang et al. describe the production of transistors by inkjet printing processes. The organic semiconductor used was α,α′-dihexylquaterthiophene. The mobilities found here of 0.043 cm²/Vs correspond to those of layers of the material applied by vapour deposition. However, very small electrode separations in the transistor of 6 μm were selected. In a roll-to-roll bulk printing process, such small structures cannot be generated. Modern printing processes currently achieve resolutions of approx. 20-50 μm. At these distance, homogeneity and phase interfaces in the semiconductive layer play a significantly greater role.

Russell et al. describe, in Appl. Phys. Lett. 87 222109 (2005), the use of mixtures of poly(3-hexylthiophene-2,5-diyl) and α,α′-dihexylquaterthiophene for semiconductor layers in organic field-effect transistors. In this case, the α,α′-dihexylquaterthiophene forms crystalline islands connected by the polymer. The mobilities found for the semiconductor layer are, however, limited by the lower mobilities of the poly(3-hexylthiophene-2,5-diyl) compared to α,α′-dihexylquaterthiophene. In Jap. J. Appl. Phys. (2005), Volume 44, p. L1567, mixtures of poly(3-hexylthiophene-2,5-diyl) and α,α′-dihexylsexithiophene are used to produce field-effect transistors. In order to achieve adequate solubility of the compounds, the solutions, however, have to be heated to 190° C., which is unsuitable for an industrial application. Adv. Funct. Mater. 2007, 17, 1617-1622 describes a cyclohexyl-substituted quarterthiophene, which crystallizes from oversaturated solution. However, this method of processing does not allow mass production. In addition, small channel lengths have to be used in the electrode structure, in order to ensure that the crystals have sufficient overlap over these structures. The production of such small electrode structures again requires complicated lithographic methods, which cannot be used in a rapid printing process for mass production.

There was thus a need for semiconductors which, after processing from solvents, have improved properties.

The object of the invention consists in providing organic compounds which can be processed from common solvents which give rise to semiconductive films with good properties and which remain sufficiently stable in the course of storage under air. Such compounds would be outstandingly suitable for large-area application of organic semiconductive layers.

It would be especially desirable for the compounds to form high-value layers of uniform thickness and morphology and to be suitable for electronic applications.

It has been found that, surprisingly, oligomeric organic compounds and mixtures thereof with macromolecular compounds having a core-shell structure and/or compounds comprising monomeric linear compounds give rise to particularly suitable films with particular properties. In this case, the oligomeric compounds consist of compounds with a linear structure connected to one another via flexible bridges. This is surprising and entirely unexpected, since compounds with high purity and uniformity have been pursued as far as possible to date in order to positively influence the crystallization behaviour.

In particular, the film morphology and the resulting macroscopic electrical properties of the films of oligomeric organic compounds and mixtures thereof with macromolecular compounds having a core-shell structure and/or compounds comprising monomeric linear compounds are improved over the semiconductors formed from pure monomeric linear compounds or from pure macromolecular compounds with core-shell structure.

The invention provides oligomeric compounds of the general formula (M)

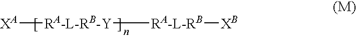

where
L is a linear conjugated oligomeric chain, preferably one containing optionally substituted thiophene, phenylene or fluorenyl units,
R^{A}, R^{B} are each independently identical or different, linear or branched C₂-C₂₀-alkylene radicals, C₃-C₈-cycloalkylene radicals, mono- or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals, preferably linear or branched C₂-C₂₀-alkylene radicals,
X^{A}, X^{B} are each independently identical or different groups selected from an optionally substituted vinyl group, a chlorine group, iodine group, hydroxyl group, alkoxy group having 1 to 3 carbon atoms, alkoxysilyl group, silyl group, chlorosilyl group, siloxane group, carboxyl group, methyl or ethyl carbonate group, aldehyde group, methyl-carbonyl group, amino group, amido group, sulphone group, sulphonic acid group, halosulphonyl group, sulphonate group, phosphonic acid group, phosphonate group, trichloromethyl group, tribromomethyl group, cyanate group, isocyanate group, thiocyanate group, isothiocyanate group, cyano group, nitro group or H, preferably disiloxane or H,
Y are each independently identical or different groups, but preferably identical groups selected from an optionally substituted alkoxysilylene group, silylene group, chlorosilylene group, methylene group, dichloromethylene group, dibromomethylene group or a divalent siloxane group, disiloxane group, carboxyl group, carbonate group, amino group, amido group, sulphate group, phosphonate group, phosphate group or borate group or S or O, preferably an ether group or a divalent disiloxane group, and
n is an integer of 1 to 10, preferably an integer of 1 to 3.

The invention further provides mixtures comprising at least one compound of the general formula (M) and at least one macromolecular compound with core-shell structure, the core (K) having a macromolecular base structure based on silicon and/or carbon and being bonded via a connecting chain based on carbon to at least three carbon-based linear oligomeric chains having conjugated double bonds throughout, and the linear conjugated chains each being saturated with at least one further chain, especially aliphatic, araliphatic or oxyaliphatic chain without conjugated double bonds.

The inventive embodiment is that of mixtures comprising the oligomeric compound(s) of the general formula (M) and the macromolecular compound(s) with core-shell structure in a weight ratio of 99:1 to 1:99, preferably 10:90 to 90:10. Such mixtures may either be solid mixtures or else solutions comprising the two aforementioned components.

The invention still further provides mixtures comprising at least one oligomeric compound of the general formula (M) and at least one compound of the general formula (P)

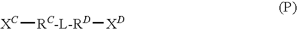

where
L is a linear conjugated oligomeric chain, preferably one containing optionally substituted thiophene, phenylene or fluorenyl units,
R^{C}, R^{D} are each independently identical or different, linear or branched C₂-C₂₀-alkylene radicals, C₃-C₈-cycloalkylene radicals, mono or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals, preferably linear or branched C₂-C₂₀-alkylene radicals,
X^{C}, X^{D} are each independently identical or different groups selected from an optionally substituted vinyl group, a chlorine group, iodine group, hydroxyl group, alkoxy group having 1 to 3 carbon atoms, alkoxysilyl group, silyl group, chlorosilyl group, siloxane group, carboxyl group, methyl or ethyl carbonate group, aldehyde group, methyl-carbonyl group, amino group, amido group, sulphone group, sulphonic acid group, halosulphonyl group, sulphonate group, phosphonic acid group, phosphonate group, trichloromethyl group, tribromomethyl group, cyanate group, isocyanate group, thiocyanate group, isothiocyanate group, cyano group, nitro group or H, preferably disiloxane or H.

The inventive embodiment is that of mixtures comprising the oligomeric compound(s) of the general formula (M) and the compound(s) of the general formula (P) in a weight ratio of 99:1 to 1:99, preferably 20:80 to 80:20. Such mixtures may either be solid mixtures or else solutions comprising the two aforementioned components.

In the case that the abovementioned inventive mixtures, i.e. mixtures comprising at least one oligomeric compound of the general formula (M) and at least one compound of the general formula (P) or at least one macromolecular compound with core-shell structure, are solutions, suitable solvents are aromatics, ethers or halogenated aliphatic hydrocarbons, for example chloroform, toluene, xylenes, benzene, diethyl ether, dichloromethane, chlorobenzene, dichlorobenzene or tetrahydrofuran, or mixtures thereof.

The organic macromolecular compounds with core-shell structure may, in a preferred embodiment, be oligomers or polymers. In the context of the invention, oligomers are understood to mean compounds having a molecular weight below 1000 daltons, and polymers to mean compounds having a mean molecular weight of 1000 daltons and higher. The mean molecular weight may, according to the test method, be the number average (Mₙ) or weight average (M_{w}). What is meant here is the number average (Mₙ).

In the context of the invention, the core-shell structure is a structure at the molecular level, i.e. it is based on the construction of a molecule as such.

The terminal linkage site of the linear conjugated oligomeric chain is understood to mean the site in the terminal unit of the linear oligomeric chain with conjugated double bonds through which there is no further linkage of a further unit. “Terminal” should be understood in the sense of furthest removed from the core. The linear oligomeric chain with conjugated double bonds throughout will subsequently also be referred to as linear conjugated oligomeric chain for short.

The macromolecular compounds with core-shell structure preferably have a core-shell structure of the general formula (Z)

K-[-V-L-R]_{q} (Z)

in which
K is an n-functional core,
V is a connecting chain,
L is a linear conjugated oligomeric chain,
R are linear or branched C₂-C₂₀-alkyl radicals, C₃-C₈-cycloalkylene radicals, mono- or polyunsaturated C₂-C₂₀-alkenyl radicals, C₂-C₂₀-alkoxy radicals, C₂-C₂₀-aralkyl radicals or C₂-C₂₀-oligoether or C₂-C₂₀-polyether radicals,
q is an integer greater than or equal to 3, preferably equal to 4.

In this structure, the shell of the preferred compounds is formed from the q-V-L-R units, each of which are connected to the core.

When, for example, q is 3, 4 or 6, these are structures of the formulae (Z-3), (Z-4) or (Z-6)

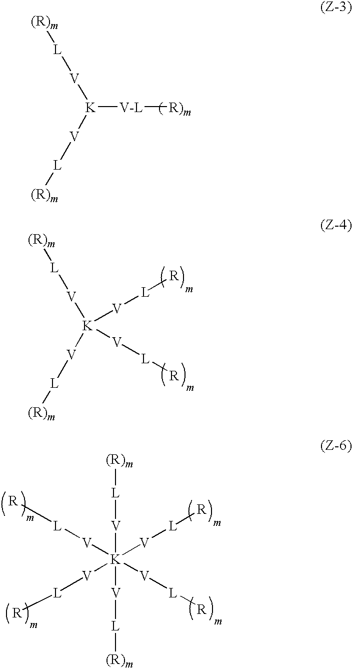

in which K, V, L and R are each as defined above.

Such compounds are constructed such that a core formed from polyfunctional units, i.e. a branched core, connecting chains, linear conjugated oligomeric chains and nonconjugated chains are bonded to one another.

The core formed from polyfunctional units preferably has dendritic or hyperbranched structures.

Hyperbranched structures and their preparation are known per se to those skilled in the art. Hyperbranched polymers or oligomers have a particular structure predetermined by the structure of the monomers used. The monomers used are so-called ABn monomers, i.e. monomers which bear two different functionalities A and B. Among these, one functionality (A) is present only once per molecule, the other functionality (B) more than once (n times). The two functionalities A and B may be reacted with one another to form a chemical bond, for example polymerized. Owing to the monomer structure, the polymerization forms branched polymers with a tree-like structure, known as hyperbranched polymers Hyperbranched polymers have no regular branching sites, no rings and virtually exclusively B functionalities at the chain ends. Hyperbranched polymers, their structure, the question of branching and their nomenclature is described for the example of hyperbranched polymers based on silicones in L. J. Mathias, T. W. Carothers, Adv. Dendritic Macromol. (1995), 2, 101-121 and the studies cited therein.

In the context of the invention, the hyperbranched structures are preferably dendritic polymers.

In the context of the invention, dendritic structures are synthetic macromolecular structures which are formed stepwise by linking two or more monomers in each case to each already bonded monomer, such that the number of monomer end groups grows exponentially with each step and, at the end, a spherical tree structure is formed. In this way, three-dimensional macromolecular structures with groups which have branching points and extend from a centre in a regular manner up to the periphery are formed. Such structures are typically formed layer by layer by processes known to those skilled in the art. The number of layers is typically referred to as generations. The number of branches in each layer, and also the number of terminal groups, increases with increasing generations. Owing to their regular structure, dendritic structures can offer particular advantages. Dendritic structures, preparation methods and nomenclature are known to those skilled in the art and are described, for example, in G. R. Newkome et al., Dendrimers and Dendrons, Wiley-VCH, Weinheim, 2001.

The structures usable in the core formed from dendritic or hyperbranched structures, also referred to hereinafter as the dendritic or hyperbranched core for short, are, for example, those which are described in U.S. Pat. No. 6,025,462. These are, for example, hyperbranched structures such as polyphenylenes, polyether ketones, polyesters as described, for example, in U.S. Pat. No. 5,183,862, U.S. Pat. No. 5,225,522 and U.S. Pat. No. 5,270,402, aramids as described, for example, in U.S. Pat. No. 5,264,543, polyamides as described, for example, in U.S. Pat. No. 5,346,984, polycarbosilanes or polycarbosiloxanes as described, for example, in U.S. Pat. No. 6,384,172, or polyarylenes as described, for example, in U.S. Pat. No. 5,070,183 or U.S. Pat. No. 5,145,930, or dendritic structures, for example polyarylenes, polyarylene ethers or polyamidoamines as described, for example, in U.S. Pat. No. 4,435,548 and U.S. Pat. No. 4,507,466, and also polyethyleneimines as described, for example, in U.S. Pat. No. 4,631,337.

However, it is also possible to use other structural units to form the dendritic or hyperbranched core. The role of the dendritic or hyperbranched core consists predominantly in providing a number of functionalities and hence of forming a matrix to which the connecting chains with the linear conjugated oligomeric chains are joined and can thus be arranged in a core-shell structure. The linear conjugated oligomeric chains are prepositioned by joining them to the matrix and thus increase their effectiveness.

The dendritic or hyperbranched core has a series of functionalities—in the sense of linkage sites—which are suitable for attachment of the connecting chains to the linear conjugated oligomeric chains. In particular, the dendritic core, just like the core formed from hyperbranched structures, has at least three, but preferably at least four functionalities.

Preferred structures in the dendritic or hyperbranched core are 1,3,5-phenylene units (formula V-a) and units of the formulae (V-b) to (V-e), where a plurality of identical or different units of the formulae (V-a) to (V-e) are bonded to one another

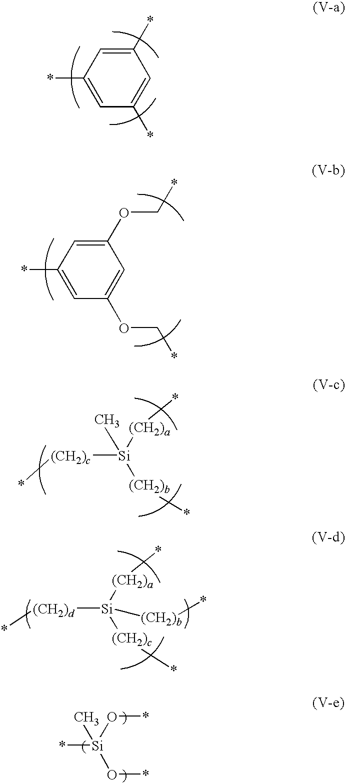

where, in the units of the formulae (V-c) and (V-d), a, b, c and d are each independently 0, 1, 2 or 3.

The positions indicated by * in the formulae (V-a) to (V-e) and in further formulae used below indicate the linkage sites. The units (V-a) to (V-e) are bonded to one another via these or to the linear conjugated oligomeric chains (L) via the connecting chains.

Examples of dendritic cores (K) formed from units of the formula (V-a) are the following:

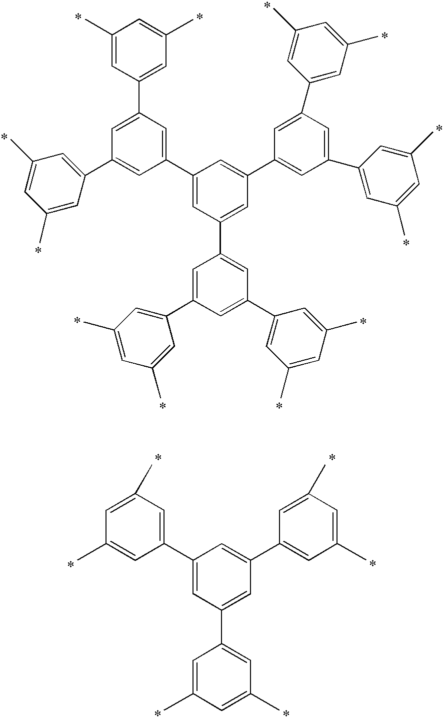

At the positions indicated by *, there is linkage via the connecting chains (V) to the linear conjugated oligomeric chains (L).

The shell of the macromolecular compounds with core-shell structure is formed from connecting chains (V), linear conjugated oligomeric chains (L) and the nonconjugated chains (R). The oligomeric compounds of the general formula (M) are formed from the linear conjugated oligomeric chains (L) and the nonconjugated chains (R) with a terminal functionality (X) and a bridging unit (Y). The compounds of the general formula (P) are formed from the linear conjugated chains (L) and the nonconjugated chains (R) with a terminal functionality (X).

Bonding chains (V) are preferably those which have a high flexibility, i.e. a high (intra)molecular mobility, and as a result bring about a geometric arrangement of the -L-R segments about the core K. In the context of the invention, “flexible” should be understood in the sense of (intra)molecularly mobile.

Suitable connecting chains are in principle linear or branched chains which have the following structural features:
carbon atoms bonded to carbon atoms by single bonds,
hydrogen atoms bonded to carbon,
oxygen atoms bonded to carbon by single bonds,
silicon atoms bonded to carbon by single bonds and/or
silicon atoms bonded to oxygen by single bonds,
   which are preferably formed from a total of 6 to 60 atoms and preferably do not contain any ring structures.

Suitable connecting chains are more preferably linear or branched C₂-C₂₀-alkylene chains, for example ethylene, n-butylene, n-hexylene, n-octylene and n-dodecylene chains, linear or branched polyoxyalkylene chains, for example oligoether chains containing —OCH₂—, —OCH(CH₃)— or —O—(CH₂)₄— segments, linear or branched siloxane chains, for example those containing dimethylsiloxane structural units and/or straight-chain or branched carbosilane chains, i.e. chains which contain silicon-carbon single bonds, where the silicon and the carbon atoms may be arranged in an alternating, random or blockwise manner in the chains, for example those with —SiR₂—CH₂—CH₂—CH₂—SiR₂-structural units.

Suitable linear conjugated oligomeric chains (L) of the general formulae (M), (P) and (Z) are in principle all chains which have structures which form oligomers or polymers which are electrically conductive or semiconductive as such. These are, for example, optionally substituted polyanilines, polythiophenes, polyethylenedioxythiophenes, polyphenylenes, polypyrroles, polyacetylenes, polyisonaphthenes, polyphenylenevinylenes, polyfluorenes, which may be used as homopolymers or -oligomers or as copolymers or -oligomers. Examples of such structures, which may be used with preference as linear conjugated oligomeric chains, are chains composed of 2 to 10, more preferably 2 to 8 units of the general formula (VI-a) to (VI-f)

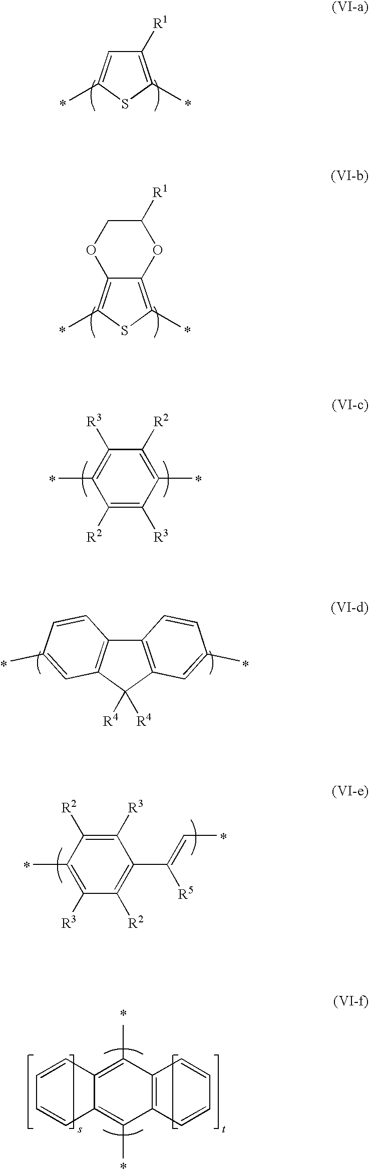

where
R¹, R² and R³ may be the same or different and are each hydrogen, straight-chain or branched C₁-C₂₀-alkyl or C₁-C₂₀-alkoxy groups, and are preferably the same and are each hydrogen,
R⁴ may be the same or different and are each hydrogen, straight-chain or branched C₁-C₂₀-alkyl groups or C₁-C₂₀-alkoxy groups, preferably hydrogen or C₆-C₁₂-alkyl groups and
R⁵ is hydrogen or a methyl or ethyl group, preferably hydrogen, and
s, t are each independently an integer from 0 to 4 and s+t≧3, preferably s+t=4.

The positions indicated by * in the formulae (V-a) to (V-f) indicate the linkage sites through which the units (V-a) to (V-f) are linked to one another to form the linear conjugated oligomeric chain or bear the nonconjugated chains (R) at the particular chain ends.

Particular preference is given to linear conjugated oligomeric chains which comprise units formed from optionally substituted 2,5-thiophenes (VI-a) or (VI-b) or optionally substituted 1,4-phenylenes (VI-c). The preceding numbers 2,5- and 1,4- specify the linkage positions in the units.

Here and hereinafter, unless stated otherwise, “substituted” is understood to mean substitution by alkyl, especially by C₁-C₂₀-alkyl groups, or by alkoxy, especially by C₁-C₂₀-alkoxy groups.

Very particular preference is given to linear conjugated oligomeric chains with units formed from optionally substituted 2,5-thiophenes (VI-a) or 2,5-(3,4-ethylenedioxythiophenes) (VI-b).

The linear conjugated oligomeric chains, indicated by L in the general formulae (M), (P) and (Z), are each saturated by a nonconjugated chain (R) at the terminal linkage sites. Nonconjugated chains are preferably those which have a high flexibility, i.e. a high (intra)molecular mobility, interact well with solvent molecules as a result and thus provide improved solubility. In the context of the invention “flexible” should be understood in the sense of (intra) molecularly mobile. The nonconjugated chains (R) are optionally oxygen-interrupted straight-chain or branched aliphatic, unsaturated or araliphatic chains having 2 to 20 carbon atoms, preferably having 6 to 20 carbon atoms, or C₃-C₈-cycloalkylene. Preference is given to aliphatic and oxyaliphatic groups, i.e. alkoxy groups or oxygen-interrupted straight-chain or branched aliphatic groups such as oligo- or polyether groups, or C₃-C₈-cycloalkylene. Particular preference is given to unbranched C₂-C₂₀-alkyl or C₂-C₂₀-alkoxy groups or C₃-C₈-cycloalkylene. Examples of suitable chains are alkyl groups such as n-hexyl, n-heptyl, n-octyl, n-nonyl, n-decyl and n-dodecyl groups, and alkoxy groups such as n-hexyloxy, n-heptyloxy, n-octyloxy, n-nonyloxy, n-decyloxy and n-dodecyloxy groups, or C₃-C₈-cycloalkylene such as cyclopentyl, cyclohexyl or cycloheptyl.

Examples of structural elements L-R of the general formula (Z) composed of linear conjugated oligomeric chains which are each saturated at the terminal linkage site by a nonconjugated chain include structural elements of the general formulae (VI-a-R) and (VI-b-R):

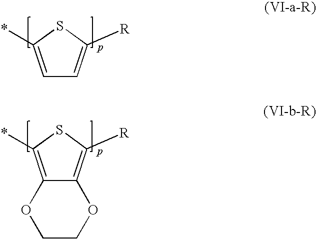

in which R is as defined above for the general formula (Z) and
p is an integer of 2 to 10, preferably of 2 to 8, more preferably 2 to 7.

Examples of structural elements -R^{A}-L-R^{B}- of the general formula (M) include structural elements of the general formulae (VI-a-R^{A/B}) and (VI-b-R^{A/B}):

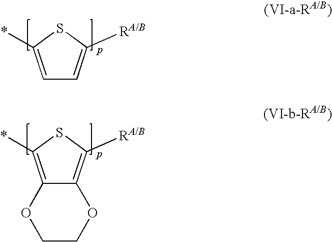

in which R^{A} and R^{B} are each as defined above for the general formula (M) and
p is an integer of 2 to 10, preferably of 2 to 8, more preferably of 4 to 6.

Preferred embodiments of the macromolecular compounds with core-shell structure are core-shell structures which contain, in the dendritic core, siloxane and/or carbosilane units, as the connecting chain linear unbranched alkylene groups, as linear conjugated oligomeric chains unsubstituted oligothiophene chains and/or oligo(3,4-ethylenedioxythiophene) chains having 2 to 8, preferably 4 to 6, optionally substituted thiophene or 3,4-ethylenedioxythiophene units, and C₆-C₁₂-alkyl groups as flexible nonconjugated chains.

Examples of these include the following compounds of the formulae (Z-4-a) and (Z-4-b):

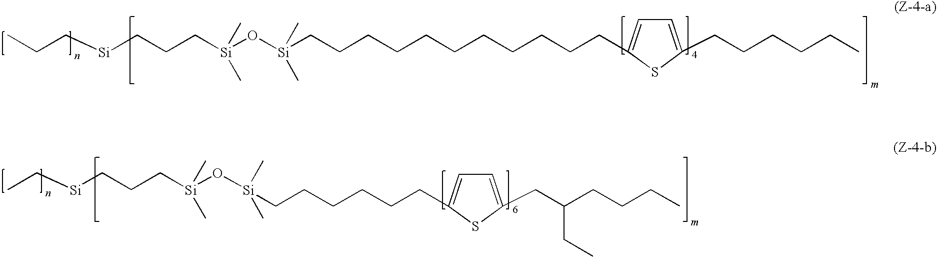

where, in (Z-4-a) and (Z-4-b), n is 0 or 1 and m is 3 or 4, with the condition that n+m=4; preferably, n is 0 and m is 4.

Preferred embodiments of the oligomeric compounds of the general formula (M) are those which contain, as linear conjugated oligomeric chains (L), optionally substituted thiophene units, more preferably optionally substituted oligothiophene chains and/or oligo(3,4-ethylenedioxythiophene) chains having 2 to 8 optionally substituted thiophene or 3,4-ethylenedioxythiophene units of the general formula (VI-a) or (VI-b), more preferably 4 to 6 thiophene or 3,4-ethylenedioxythiophene units of the general formula (VI-a) or (VI-b). In a further preferred embodiment of the invention, the oligomeric compounds of the general formula (M) contain, as nonconjugated chains R^{A} and R^{B}, identical or different, linear or branched C₁-C₁₂-alkylene groups, and, as the terminal X^{A} and X^{B} group, hydrogen. In yet a further preferred embodiment of the invention, Y in the oligomeric compounds of the general formula (M) is a carbonate, ether or divalent disiloxane group, more preferably an ether or divalent disiloxane group. A very particularly preferred embodiment of the invention comprises oligomeric compounds of the general formula (M) in which n is 1.

Examples of these include the following compounds of the formulae (M-V) to (M-VIII):

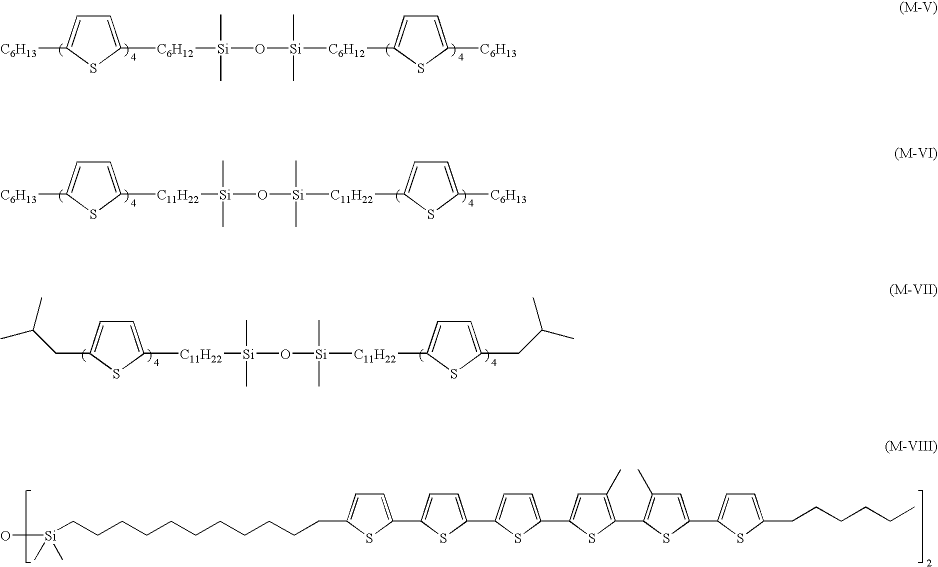

Preferred embodiments of the present invention are mixtures comprising one or more compounds of the general formula (Z) and one or more compounds of the general formula (M) in a weight ratio of 1:99 to 99:1, preferably 10:90 to 90:10, in which the components of the formulae (Z) and (M) have linear oligomeric chains (L) with identical units, preferably linear oligomeric chains with 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b). Particular preference is given to mixtures comprising one or more compounds of the general formula (Z) and one or more compounds of the general formula (M) in a weight ratio of 1:99 to 99:1, preferably 10:90 to 90:10, in which the components of the formulae (Z) and (M) contain linear oligomeric chains (L) with the same number of identical units in the oligomeric chains, preferably those linear oligomeric chains having 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b).

Preferred embodiments of the monomeric linear compounds of the general formula (P) are those which contain, as linear conjugated oligomeric chains (L), optionally substituted thiophene units, preferably optionally substituted oligothiophene chains and/or oligo(3,4-ethylenedioxythiophene) chains and/or thiophene-phenylene chains having 2 to 8 aromatic units of the general formula (VI-a), (VI-b) or (VI-c), most preferably 4 to 6 aromatic units consisting of thiophene or 3,4-ethylenedioxythiophene or thiophene-phenylene chains of the general formula (VI-a), (VI-b) or (VI-c). In a further preferred embodiment of the invention, the compounds of the general formula (P) contain, as nonconjugated chains R^{C} and R^{D}, identical or different, linear or branched C₁-C₁₂-alkylene groups and, as the terminal X^{C} and X^{D} group, hydrogen.

Examples of these include the following compounds of the formulae (P-I) to (P-III):

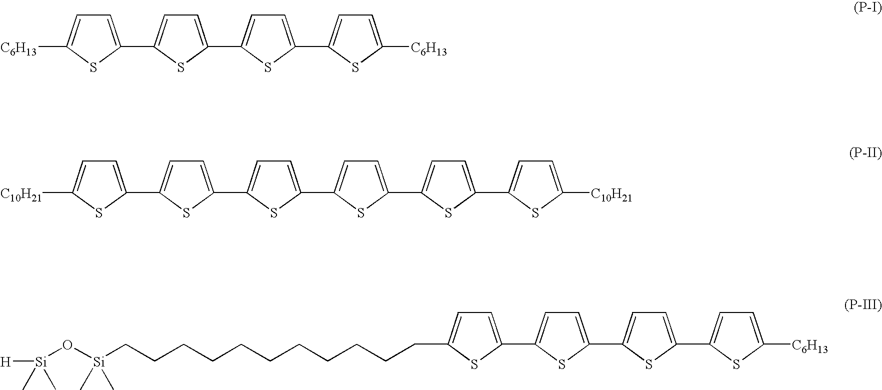

The compounds P-I, P-II and P-III are, for example, known from Chem. Mater. (1998), Volume 10, p. 457, J. Mater. Chem. (2003), Volume 13, p. 197, J. Am. Chem. Soc. (2005), Volume 127, p. 1348 and EP-A 1 531 155.

A preferred embodiment of the present invention is that of mixtures comprising one or more compounds of the general formula (M) and one or more compounds of the general formula (P) in a weight ratio of 1:99 to 99:1, preferably 20:80 to 80:20, in which the components of the formulae (M) and (P) have linear oligomeric chains (L) with identical units, preferably linear oligomeric chains having 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b). Particular preference is given to mixtures comprising one or more compounds of the general formula (M) and one or more compounds of the general formula (P) in a weight ratio of 1:99 to 99:1, preferably 20:80 to 80:20, in which the components of the formulae (M) and (P) have linear oligomeric chains (L) having the same number of identical units in the oligomeric chains, preferably those linear oligomeric chains having 2 to 8, more preferably 4 to 6, thiophene or 3,4-ethylenedioxythiophene units of the general formula (VI-a) or (VI-b).

The invention further provides mixtures comprising at least one oligomeric compound of the general formula (M), at least one macromolecular compound with core-shell structure and at least one compound of the general formula (P). For the particular compounds of the general formula (M), (P) and (Z), the abovementioned preferred ranges apply.

Preferred embodiments of the present invention are mixtures comprising one or more compounds of the general formula (Z), one or more compounds of the general formula (M) and one or more compounds of the general formula (P) in a weight ratio of 1:99 to 99:1, preferably 10:90 to 90:10, in which the components of the formulae (Z), (M) and (P) have linear oligomeric chains (L) with identical units, preferably linear oligomeric chains having 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b). Particular preference is given to mixtures comprising one or more compounds of the general formula (Z), one or more compounds of the general formula (M) and one or more compounds of the general formula (P) in a weight ratio of 1:99 to 99:1, preferably 10:90 to 90:10, in which the components of the formulae (Z), (M) and (P) have linear oligomeric chains (L) with the same number of identical units in the oligomeric chains, preferably those linear oligomeric chains having 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b). The abovementioned weight ratios in an inventive mixture should, for example, be understood such that one or more compounds of the general formula (Z) and one or more compounds of the general formula (M) together form a proportion by weight of 1 and one or more compounds of the general formula (P) form 99 parts by weight of the mixture.

The invention still further provides mixtures comprising at least one compound of the general formula (P) and at least one macromolecular compound with core-shell structure, the core having a macromolecular base structure based on silicon and/or carbon and being connected via a connecting chain based on carbon to at least three carbon-based linear oligomeric chains having conjugated double bonds throughout, and the linear conjugated chains each being saturated with at least one further chain, especially aliphatic, araliphatic or oxyaliphatic chain without conjugated double bonds. In this case, the abovementioned preferred ranges apply to the particular compounds of the general formula (P) and (Z).

Preferred embodiments of the present invention are mixtures comprising one or more compounds of the general formula (Z) and one or more compounds of the general formula (P) in a weight ratio of 1:99 to 99:1, preferably 20:80 to 80:20, in which the components of the formulae (Z) and (P) have linear oligomeric chains (L) with identical units, preferably linear oligomeric chains with 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b). Particular preference is given to mixtures comprising one or more compounds of the general formula (Z) and one or more compounds of the general formula (M) and (P) in a weight ratio of 1:99 to 99:1, preferably 20:80 to 80:20, in which the components of the formulae (Z), (M) and (P) have linear oligomeric chains (L) with the same number of identical units in the oligomeric chains, preferably those linear oligomeric chains having 2 to 8 thiophene or 3,4-ethylenedioxythiophene units of the general formula (VI-a) or (VI-b), most preferably 4 to 6 thiophene or 3,4-ethylenedioxythiophene units of the general formula (VI-a) or (VI-b).

Layers formed from the inventive oligomeric compounds of the general formula (M) and layers formed from the inventive mixtures comprising the oligomeric compounds of the general formula (M) and the macromolecular compounds with core-shell structure and/or the monomeric linear compounds of the general formula (P) and layers comprising the macromolecular compounds with core-shell structure and monomeric linear compounds of the general formula (P) are preferably conductive or semiconductive. The invention more preferably provides layers of the compounds or mixtures which are semiconductive. Particular preference is given to those layers of the compounds or mixtures which have a mobility for charge carriers of at least 10⁻⁴ cm²/Vs. Charge carriers are, for example, positive hole charges.

The inventive compounds and mixtures are typically very soluble in common organic solvents and are thus outstandingly suitable for processing from solution. Especially suitable solvents are aromatics, ethers or halogenated aliphatic hydrocarbons, for example chloroform, toluene, benzene, xylenes, diethyl ether, dichloromethane, chlorobenzene, dichlorobenzene or tetrahydrofuran, or mixtures thereof. What is surprising is in particular that the inventive oligomeric compounds of the general formula (M) and the mixtures comprising the inventive compounds of the general formula (M) and the macromolecular compounds with core-shell structure and/or the compounds of the general formula (P), compared to the corresponding monomeric compounds, form homogeneous films which wet large areas and have large monocrystalline regions. It is also surprising that the mixing of the components does not disrupt the internal order and morphology of the layers. The inventive compounds and mixtures are therefore very suitable for large-area coating and have correspondingly good semiconductive properties in these films. The inventive semiconductive compounds or mixtures also have an outstanding thermal stability and good ageing behaviour.

The inventive oligomeric compounds of the general formula (M) and mixtures thereof with macromolecular compounds with core-shell structure and/or compounds of the general formula (P) or mixtures of compounds of the general formula (P) with macromolecular compounds with core-shell structure, for example compared to the known monomeric compounds, also have the advantage that elevated solubility in the common organic solvents is achieved. This eases the processing of the compounds to semiconductor layers.

The preparation of the inventive compounds and mixtures is possible by different process routes.

The preparation of the macromolecular compounds with core-shell structure is described in EP-A 1 580 217 and Ponomarenko et al. Chem. Mater. 2006, Volume 18, p. 4101. The preparation of the compounds of the general formula (P) is described, for example, in EP-A 1 531 155, EP-A 1 531 154, EP-A 1 439 173, WO-A 2005/080369, Kirchmeyer et al. J. Mater. Chem. 2003, Volume 13, p. 197 or Ponomarenko et al. Polymer Preprints 2005, Volume 46, p. 576. The inventive oligomeric compounds of the general formula (M) can be prepared as described in Examples 1-3.

The oligomeric compounds of the general formula (M) can be mixed with the monomeric linear compounds of the general formula (P) and/or with the macromolecular compounds with core-shell structure or the macromolecular compounds with core-shell structure and the compounds of the general formula (P) can be mixed by co-dissolution in the appropriate solvent. The dissolution process is preferably effected under hot conditions. The resulting solutions are stable and processible.

Moreover, the components can be mixed by adding one component during the preparation of the other component, for example adding the monomeric compounds of the general formula (P) during the synthesis of the oligomeric compounds of the general formula (M), or adding the oligomeric compounds of the general formula (M) during the synthesis of the macromolecular compounds with core-shell structure, such that the inventive mixtures are obtained on isolation.

In addition, the inventive mixtures can also be prepared by the combination of solutions of the individual components on a suitable substrate. The substrate used may, for example, be a structured silicon wafer or a coated glass substrate, for example coated with ITO. The individual components of the inventive mixtures may, for example, be prepared by separate dropwise application, for example by an inkjet printing process, to the substrate. A further process is, for example, the preparation of the inventive mixtures by simultaneous or successive separate metering of the individual components onto the substrate in the course of spin-coating.

For the properties of the inventive compounds and mixtures, the preparation route is unimportant.

The inventive compounds and mixtures are soluble to an extent of at least 0.1% by weight, preferably at least 1% by weight, more preferably at least 5% by weight in customary solvents, for example aromatics, ethers or halogenated aliphatic hydrocarbons, for example in chloroform, toluene, benzene, xylenes, diethyl ether, dichloromethane, chlorobenzene, dichlorobenzene or tetrahydrofuran.

The inventive compounds and mixtures form, from evaporated solutions, high-value layers of uniform thickness and morphology and are therefore suitable for electronic applications.

The invention finally further provides for the use of the inventive compounds and mixtures as semiconductors in electronic components such as field-effect transistors, light-emitting components such as organic luminescent diodes, or photovoltaic cells, lasers and sensors.

The inventive compounds and mixtures are preferably used for these purposes in the form of layers.

In order to be able to ensure functionality as a semiconductor in a sensible manner, the inventive compounds and mixtures have a sufficient mobility, for example at least 10⁻⁴ cm²/Vs. Charge mobilities can be determined, for example, as described in M. Pope and C. E. Swenberg, Electronic Processes in Organic Crystals and Polymers, 2^{nd} ed., p. 709-713 (Oxford University Press, New York Oxford 1999).

For use, the inventive compounds and mixtures are applied to suitable substrates, for example to silicon wafers, polymer films or glass panes provided with electrical or electronic structures. For the application, all application methods are useful in principle. The inventive compounds and mixtures are preferably applied from the liquid phase, i.e. from solution, and the solvent is then evaporated. The application from solution can be effected by the known processes, for example by spraying, dipping, printing and knife-coating. Particular preference is given to application by spin-coating and by inkjet printing.

The layers produced from the inventive compounds and mixtures can be modified further after the application, for example by a thermal treatment, for example with passage through a liquid-crystalline phase or for structuring, for example by laser ablation.

The invention further provides electronic components comprising the inventive compounds and mixtures as semiconductors.

The examples which follow serve to illustrate the invention by way of example and should not be interpreted as a restriction.

### EXAMPLES

The compounds of the formula Z were prepared by known processes from, for example, EP-A 1 580 217 and Chem. Mater. 2006, Volume 18, p. 4101. The compounds of the formula P were synthesized by known processes from, for example, EP-A 1 439 173, J. Mater. Chem. 2003, Volume 13, p. 197, Synthesis 1993, p. 1099 or Chem. Mater. 1993, Volume 5, p. 430. The inventive compounds of the formula (M) can be prepared analogously to the syntheses as shown in Examples 1 to 3.

All reaction vessels were baked-out and flushed with nitrogen by customary protective gas techniques before use.

### OFET Preparation:

### a) Substrate for OFETs and Cleaning

p-doped silicon wafers which have been polished on one side and have a thermally grown oxide layer of thickness 300 nm (Sil-Chem) were cut into substrates of 25 mm×25 mm in size. The substrates were first cleaned carefully. The adhering silicon splinters were removed by rubbing with a cleanroom towel (Bemot M-3, Ashaih Kasei Corp.) under flowing distilled water, and then the substrates were cleaned in an aqueous 2% water/Mucasol solution in an ultrasound bath at 60° C. for 15 min. Thereafter, the substrates were rinsed with distilled water and spin-dried in a centrifuge. Immediately before the coating, the polished surface was cleaned in a UV/ozone reactor (PR-100, UVP Inc., Cambridge, GB) for 10 min.

### b) Dielectric Layer

i. As the dielectric intermediate layer, octadi-methylchlorosilane (ODMC) (Aldrich, 246859) was used. The ODMC was poured into a Petri dish, such that the bottom was just covered. The magazine in which the upright cleaned Si substrates were present was placed thereon. Everything was covered with an upended beaker, and the Petri dish was heated to 70° C. The substrates remained in the octyldimethylchlorosilane-rich atmosphere for 15 min.
ii. Hexamethyldisilazane (HMDS): The hexamethyldisilazane (Aldrich, 37921-2) used for the dielectric intermediate layer was poured into a beaker in which the magazine comprising the upright cleaned Si substrates was disposed. The silazane covered the substrates completely. The beaker was covered and heated to 70° C. on a hotplate. The substrates remained in the silazane for 24 h. Subsequently, the substrates were dried in a dry nitrogen stream.

In the examples, ODMC is used as the dielectric intermediate layer; in the case that HMDS is used as the dielectric intermediate layer, reference is made explicitly thereto.

### c) Organic Semiconductors

To apply the semiconductor layer, a solution of the compounds in a suitable solvent was prepared. In order to achieve complete dissolution of the components, the solution was placed in an ultrasound bath at 60° C. for approx. 1 min. The concentration of the solution was 0.3% by weight.

The substrate provided with the dielectric intermediate layer was placed with the polished side up into the holder of a spin-coater (Carl Süss, RC8 with Gyrset®) and heated to approx. 70° C. with a hot-air gun. Approx. 1 ml of the still-warm solution was dripped onto the surface and the solution comprising the organic semiconductor was spin-coated onto the substrate at 1200 rpm for 30 sec at an acceleration of 500 rev/sec² and open Gyrset®. The film thus obtained was dried at 70° C. on a hotplate for 3 min. The layer was homogeneous and had no opacity.

### d) Application of the Electrodes

The electrodes for source and drain were subsequently applied to this layer by vapour deposition. To this end, a shadowmask which consisted of an electroplated Ni film with 4 cutouts composed of two interlocking combs was used. The teeth of the individual combs were 100 μm wide and 4.7 mm long. The mask was placed onto the surface of the coated substrate and fixed from the back by a magnet.

In a vapour deposition unit (Univex 350, Leybold), the substrates were subjected to vapour deposition of gold. The electrode structure thus obtained had a length of 14.85 cm with a separation of 100 μm.

### e) Capacitance Measurement

The electrical capacitance of the arrangements was determined by subjecting an identically prepared substrate, but without organic semiconductor layer, to vapour deposition in parallel behind identical shadowmasks. The capacitance between the p-doped silicon wafer and the electron applied by vapour deposition was determined with a multimeter (MetraHit 18S, Gossen Metrawatt GmbH). For this arrangement, the capacitance measured was C=0.7 nF; on the basis of the electrode geometry, an areal capacitance of C=6.8 nF/cm² was found.

### f) Electrical Characterization

The characteristics were measured with the aid of two current-voltage sources (Keithley 238). One voltage source applies an electrical potential at source and drain and determines the flowing current, while the second places an electrical potential at gate and source. Source and drain were contacted with Au rods pressed on; the highly doped Si wafer formed the gate electrode and was contacted via the back side which had been scratched to free it of oxide. The characteristics were recorded and evaluated by the known processes, as described, for example, in “Organic thin-film transistors: A review of recent advances”, C. D. Dimitrakopoulos, D. J. Mascaro, IBM J. Res. & Dev. Vol. 45 No. 1 Jan. 2001.

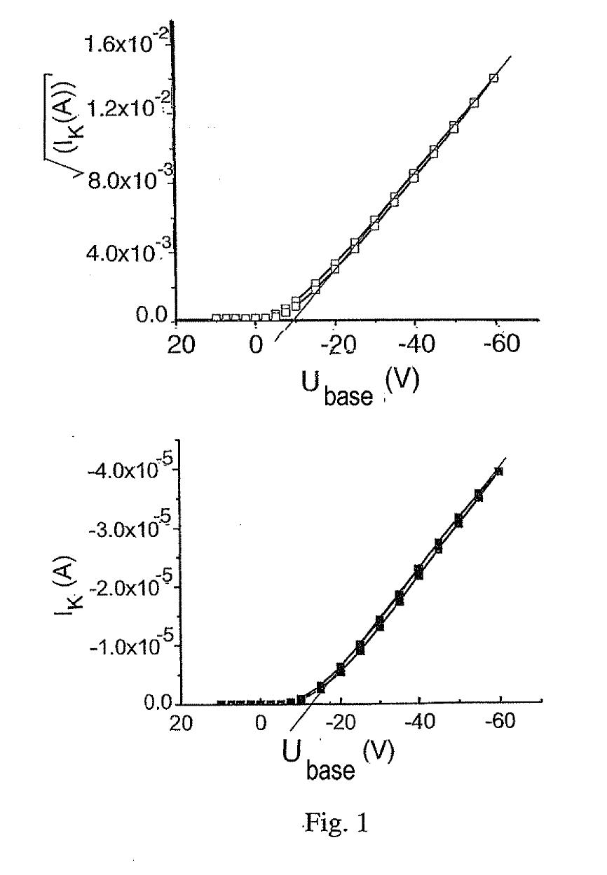

From the electrical characterization (FIG. 1), the following parameters relevant for this transistor setup were found:

i. Mobility
ii. On/Off ratio
I_{D}(U_{G}=−60V)/I_{D}(U_{G}=0V)
N.B. The sensitivity of the off current measurement I_{D}(U_{G}=0V) is limited to approx. 1 nA owing to incompletely insulated cable.
   iii. Threshold Voltage

The results of the transistor measurements are compiled in Table 1.

### Example 1

### Preparation of 1,3-bis{11-[5′″-hexyl-2,2′:5′,2″:5″, 2′″-quaterthien-5-yl]hexyl}-1,1,3,3-tetramethyldisiloxane (M-V)

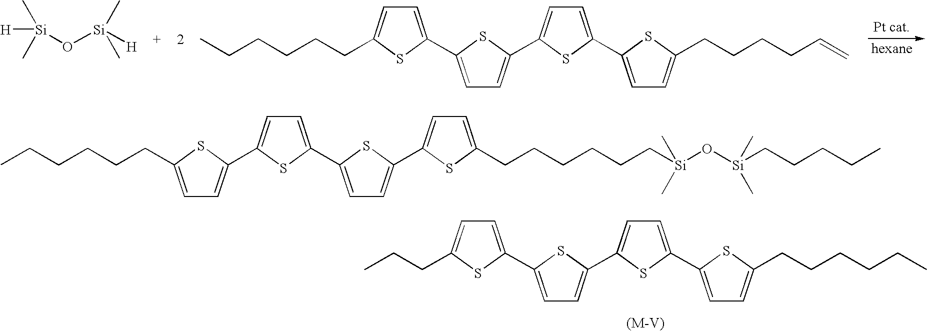

0.50 g (1.0 mmol) of 5-(hex-5-en-1-yl)-5′″-hexyl-2,2′:5′,2″:5″,2′″-quaterthiophene were dissolved in 10 ml of toluene and 80 μl of 1,1,3,3-tetramethyldisiloxane, and admixed with 10 μl of a 0.1 molar solution of 1,3-divinyl-1,1,3,3-tetramethyldisiloxane-platinum(0) in xylene. The mixture was heated to 100° C. in a closed apparatus. After 10 hours, 20 μl of 1,1,3,3-tetramethyldisiloxane were added and the mixture was stirred again at 100° C. for 10 hours. The reaction solution was applied to a chromatography column laden with silica gel and eluted with toluene. The resulting product was recrystallized from toluene.

Yield: 0.16 g (28% of theory) of orange solid (M-V).

¹H NMR (CDCl₃ TMS/ppm): 0.03 (s, 12H), 0.50 (t, 4H, J=7.3), 0.89 (t, 6H, J=7.3), 1.20-1.45 (overlapping signals, 24H), 1.67 (m, 8H, M=5, J=7.3), 2.78 (t, 8H, J=7.3), 6.66 (d, 2H, J=3.7), 6.92-7.03 (overlapping signals, 12H).

To produce OFETS, the substance was dissolved at 60° C. in a concentration of 0.3% by weight in a mixture of 1 part of toluene and 1 part of chloroform. Visually homogeneous films were obtained. The result of the electrical characterization of these OFETs (FIG. 1) is compiled in Tab. 1.

### Example 2

a) Preparation of 1,3-bis[11-(2,2′-bithien-5-yl)undecyl]-1,1,3,3-tetramethyldisiloxane (M-VI-a):

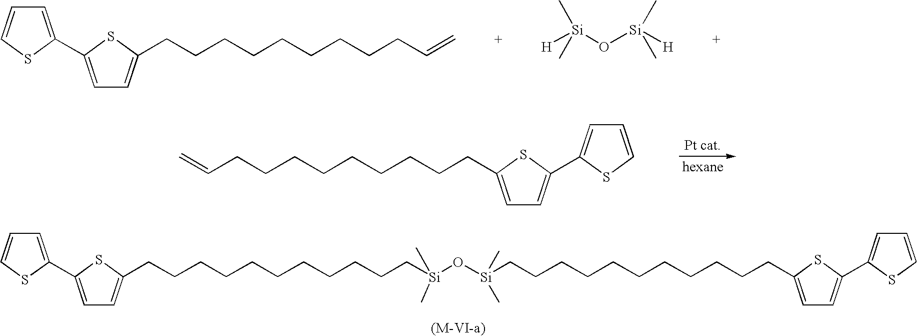

5.37 g of 11-(2,2′-bithien-5-yl)undecene were dissolved in 30 ml of anhydrous hexane. 1.42 l of tetramethyldisiloxane were added and the solution was cooled to 0° C. At this temperature, 160 μl of a 0.1 molar solution of 1,3-divinyl-1,1,3,3-tetramethyldisiloxane-platinum(0) in xylene were added. The reaction was stirred at 0° C. for 1 hour and then at room temperature for 16 hours. The reaction solution was applied to a chromatography column laden with silica gel and eluted with hexane. Yield: 5.24 g (84% of theory) of colourless to slightly yellow oil (M-VI-a).

¹H NMR (CDCl₃, TMS/ppm): 0.062 (s, 12H), 0.529 (m, 4H), 1.25-1.45 (overlapping signals with maximum at 1.296, 32H), 1.700 (m, 4H), 2.801 (t, J=7.5 Hz, 4H), 6.686 (d, J=3.4 Hz, 2H), 6.998 (m, 4H), 7.116 (d, J=5.0 Hz, 2H), 7.168 (d, J=5.0 Hz, 2H).
b) Preparation of 1,3-bis[11-(5′-bromo-2,2′-bithien-5-yl)undecyl]-1,1,3,3-tetramethyldisiloxane (M-VI-b):

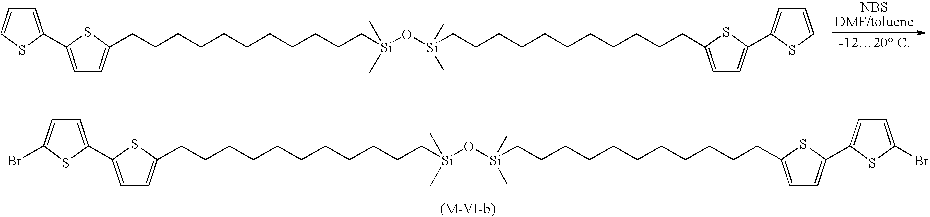

1,3-Bis[11-(2,2′-bithien-5-yl)undecyl]-1,1,3,3-tetramethyldisiloxane (M-VI-a) (9.82 g, 12.7 mmol) was dissolved in a mixture of DMF (40 ml) and toluene (40 ml), and a solution of NBS (4.55 g, 25.5 mmol) in DMF (70 ml) was added dropwise at −10° C. within 1 hour. Subsequently, a mixture was stirred at −10° C. for 30 minutes and at room temperature for 16 hours. The solution was added to water (1000 ml) and extracted by shaking with methylene chloride. The organic phase was dried over sodium sulphate, the solvent was removed and the residue was dried under high vacuum. Yield: 8.59 g (73% of theory) of slightly yellow oil (M-VI-b).

FD-MS analysis: M⁺100%, m/e=928

¹H NMR (CDCl₃, TMS/ppm): 0.031 (s, 12H), 0.498 (m, 4H), 1.23-1.44 (overlapping signals with maximum at 1.267, 32H), 1.668 (m, 4H), 2.772 (t, J=7.3 Hz, 4H), 6.659 (d, J=3.9, 2H), 6.822 (d, J=3.9, 2H), 6.907 (d, J=3.4 Hz, 2H), 6.931 (d, J=3.9, 2H).
c) Preparation of 1,3-bis{11-[5′″-hexyl 2,2′:5′,2″:5″,2′″-quaterthien-5-yl]undecyl}-1,1,3,3-tetramethyldisiloxane (M-VI):

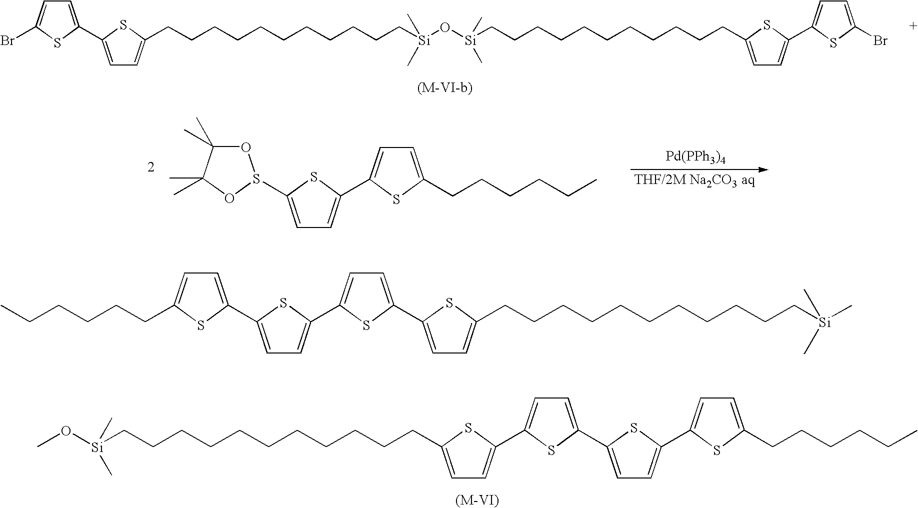

1,3-Bis[11(5′-bromo-2,2′-bithien-5-yl)undecyl]-1,1,3,3-tetramethyldisiloxane (M-VI-b) (6.32 g, 6.8 mmol) was boiled under reflux with 4,4,5,5-tetramethyl-2-[5′-hexyl-2,2′-bithien-5-yl]-1,3,2-dioxaborolan (6.13 g, 16.3 mmol) and tetrakistriphenyl-phosphinepalladium (1.54 g, 1.3 mmol) in anhydrous tetrahydrofuran (130 ml) and 85 ml of a 2M aqueous Na₂CO₃ solution for 8 hours. The reaction mixture was added to a mixture of water (300 ml) and 1M HCl (90 ml) and extracted by shaking with toluene. The organic phase was removed and dried, the solvent was removed and the residue was chromatographed (silica gel, eluant: 1:1 hexane:toluene at 55° C.). The resulting crude product was recrystallized from toluene:hexane.

Yield: 3.45 g (40% of theory) of yellow-orange powder (M-VI).

¹H NMR(CDCl₃, TMS/ppm): 0.030 (s, 12H), 0.498 (m, 4H), 0.897 (m, 6H), 1.23-1.44 (overlapping signals with maximum at 1.269, 44H), 1.681 (m, 8H), 2.787 (m, 8H), 6.675 (m, 4H), 6.974 (overlapping signals, 8H), 7.020 (d, J=3.9, 4H).

Melting point (DSC): 190° C.

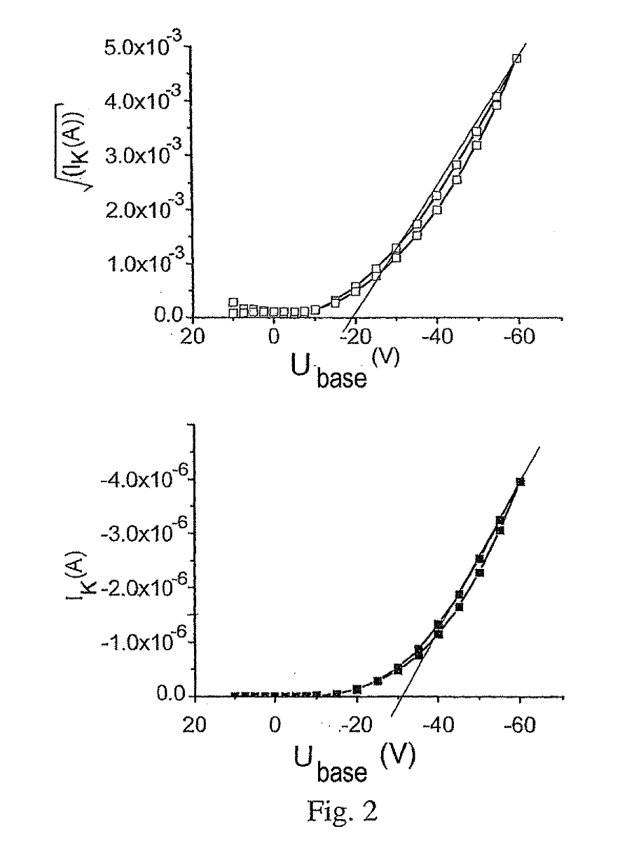

To produce OFETs, the substance was dissolved at 60° C. in a concentration of 0.3% by weight in a mixture of 1 part of toluene and 1 part of chloroform. Visually homogeneous films were obtained. The result of the electrical characterization of this OFET (FIG. 2) is compiled in Tab. 1.

### Example 3

### Preparation of 1,3-bis{11-[5′″(2-methylpropyl)-2,2′:5′,2″:5″,2′″-quaterthien-5-yl]undecyl}-1,1,3,3-tetramethyldisiloxane (M-VII)

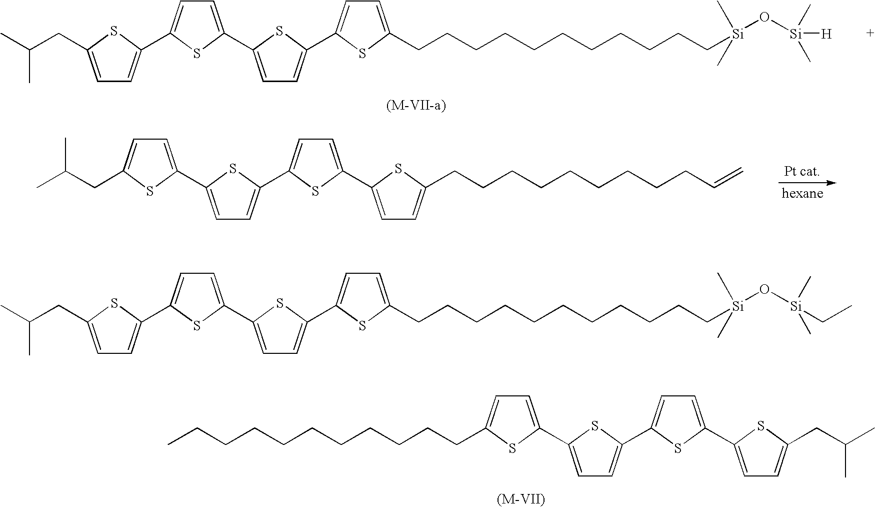

0.45 g (0.83 mmol) of 5-(2-methylpropyl)-5′″-undec-10-en-1-yl-2,2′:5′,2″:5″,2′″-quaterthiophene and 0.67 g (1.0 mmol) of 1-[11-(5′″-(2-methylpropyl)-2,2′:5′,2″:5″,2′″-quaterthien-5-yl)undecyl]-1,1,3,3-tetramethyldisiloxane were stirred in 30 ml of toluene. The reaction mixture was brought into solution by gentle heating and then degassed. 20 μl of a 0.1 molar solution of 1,3-divinyl-1,1,3,3-tetramethyldisiloxane-platinum(0) were added and the solution was heated to 75° C. After 6 hours, the reaction solution was chromatographed while warm through a column filled with silica gel (eluant: toluene, 70° C.), and the resulting product was recrystallized from toluene. Yield: 0.69 g (69% of theory) of yellow-orange powder (M-VII).

¹H NMR (CDCl₃, TMS/ppm): 0.03 (s, 12H), 0.49 (t, 4H, J=7.3), 0.96 (d, 12H, J=7.3), 1.20-1.40 (overlapping signals, 32H), 1.66 (m, 4H), 1.89 (m, 2H, J=7.3), 2.65 (d, 4H, J=7.3), 2.77 (t, 4H, J=7.3), 6.66 (d, 2H, J=3.7), 6.92-7.03 (overlapping signals, 12H).

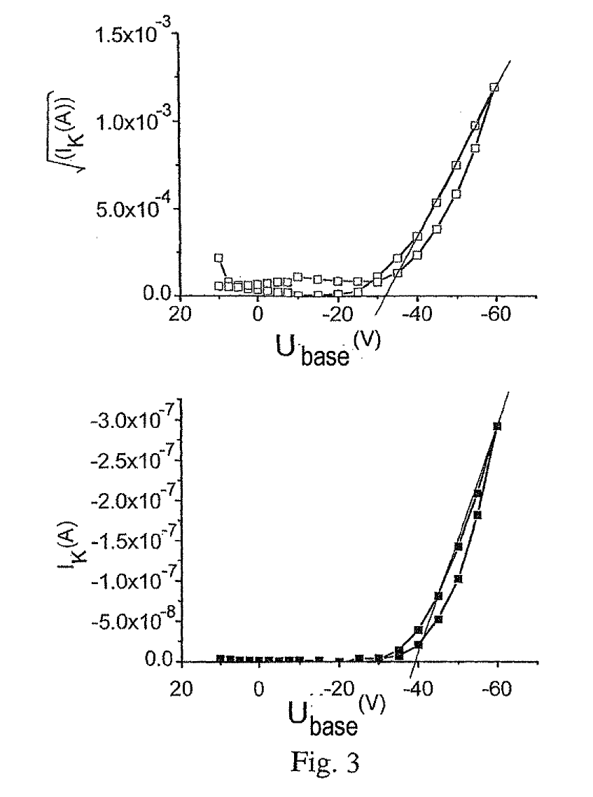

To produce OFETs, the substance was dissolved at 60° C. in a concentration of 0.3% by weight in a mixture of 1 part of toluene and 1 part of chloroform. Visually homogeneous films were obtained. The result of the electrical characterization of this OFET (FIG. 3) is compiled in Tab. 1.

### Example 4

A solution containing the compound of the formula (P-I) and the compound of the formula (M-VI) in a weight ratio of 1:2 was prepared in a mixture of 1 part by volume of chloroform and 1 part by volume of toluene. The substance mixture gave visually homogeneous films. The mixture remains stable under air even in the course of prolonged storage, as shown in FIG. 14.

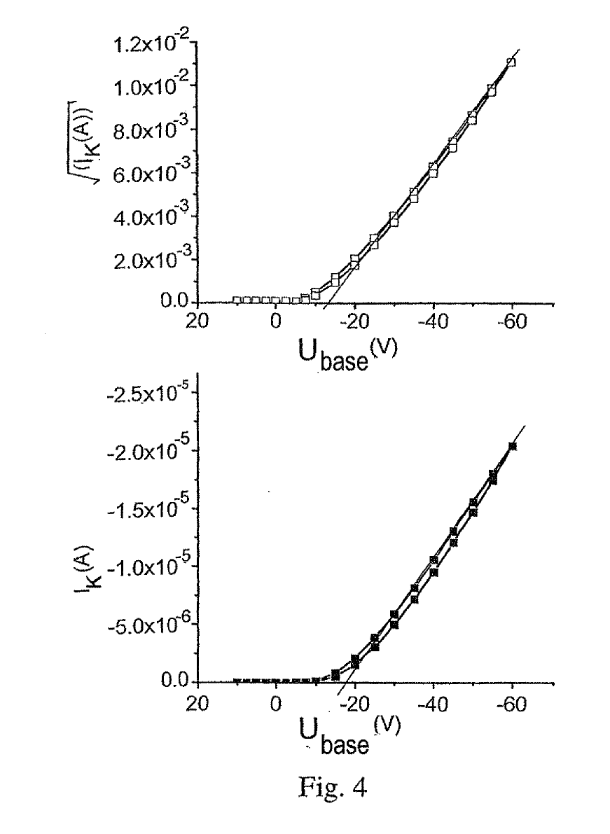

The result of the electrical characterization of this OFET (FIG. 4) is summarized in Tab. 1.

### Example 5

A solution containing the compound of the formula (P-I) and the compound of the formula (M-VII) in a weight ratio of 1:2 was prepared in a mixture of 1 part by volume of chloroform and 1 part by volume of toluene. The substance mixture gave visually homogeneous films.

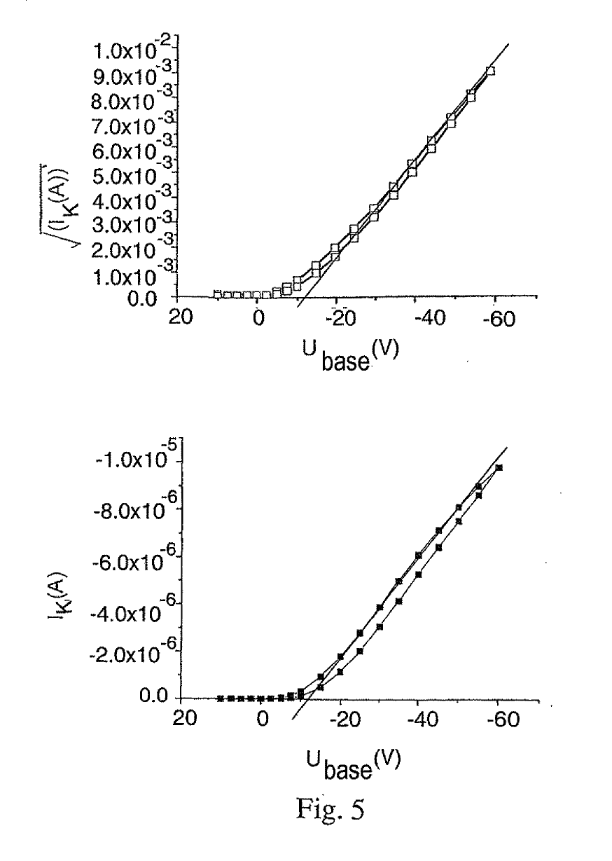

The result of the electrical characterization of this OFET (FIG. 5) is summarized in Tab. 1.

### Comparative Example 1

As an organic semiconductor, a solution of the compound of the formula (P-I) in 1 part by volume of chloroform and 1 part by volume of toluene was prepared. The film applied by spin-coating exhibited structures of individual crystals in the microscope.

No electrical characteristic could be measured.

### Example 6

As an organic semiconductor, a solution of the compound of the formula (Z-4-a-1):

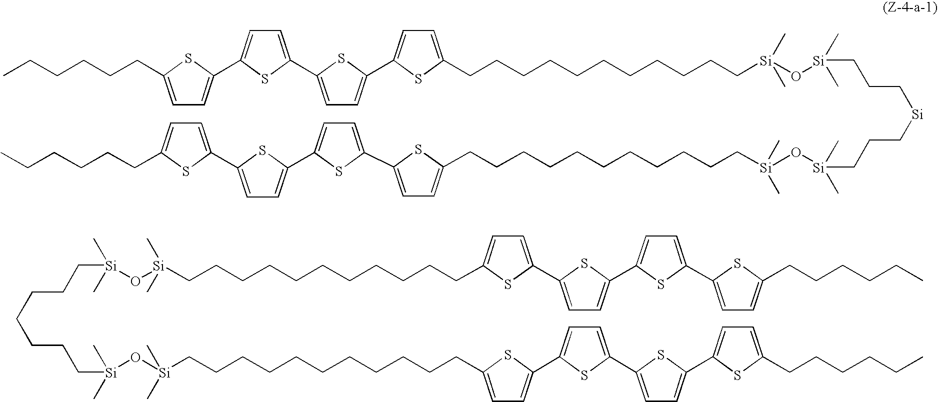

and of the compound of the formula (M-VI)

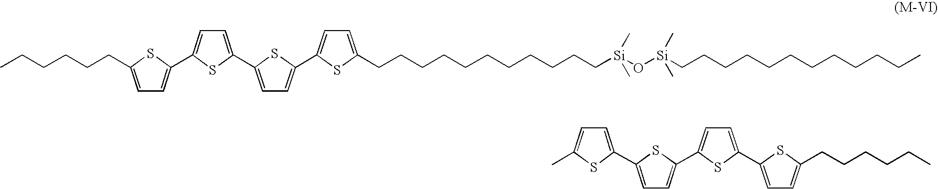

in a weight ratio of 85:15 was prepared in a solvent mixture consisting of 1 part by volume of chloroform and 1 part by volume of toluene. The concentration of the solution was 0.3% (wt.).

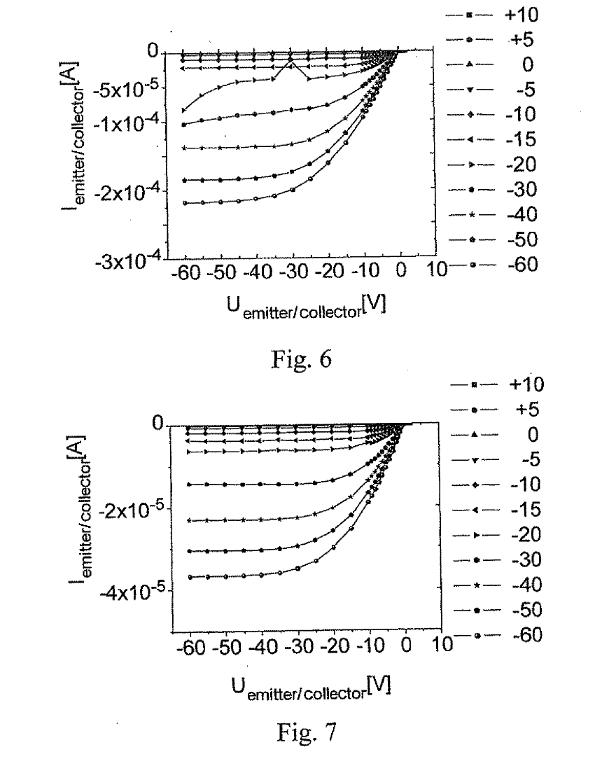

The result of the electrical characterization of this OFET (FIG. 6) is summarized in Table 1.

### Example 7

As an organic semiconductor, a solution of the compounds of the formula (Z-4-a-1), of the compound of the formula (M-VI) and of the compound of the formula (Z-4-a-2)

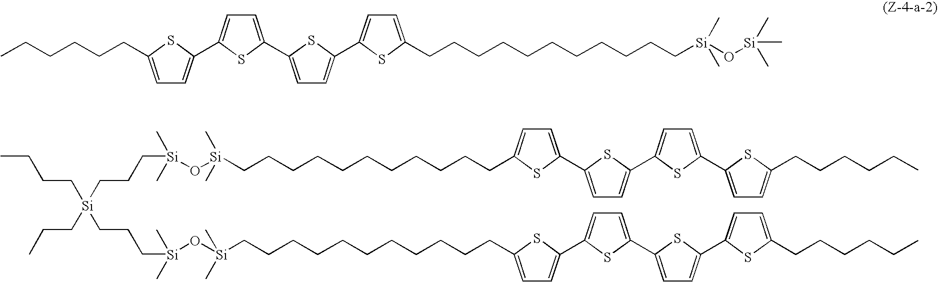

in a weight ratio of 5:4:1 was prepared in a mixture of one part by volume of chloroform and one part by volume of toluene. Films of visually good quality were obtained.

The result of the electrical characterization of this OFET (FIG. 7) is summarized in Tab. 1.

### Example 8

As an organic dielectric layer, hexamethyldisilazane was used. As an organic semiconductor, a solution of the compound (Z-4-a-1) and of the compound of the formula (P-1)

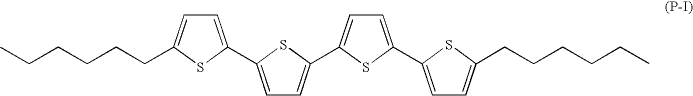

in a weight ratio of 1:1 was prepared in a mixture of one part by volume of chloroform and one part by volume of toluene. This substance mixture too gave visually homogeneous films.

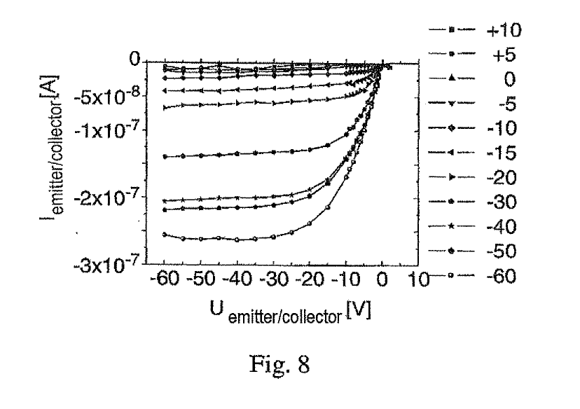

The result of the electrical characterization of this OFET (FIG. 8) is summarized in Tab. 1.

### Example 9

As an organic semiconductor, a solution of the compound (Z-4-a-1), of the compound (M-VI) and of the compound (P-I) in a weight ratio of 17:3:20 was prepared in a mixture of one part by volume of chloroform and one part by volume of toluene. Homogeneous films of visually good quality were obtained.

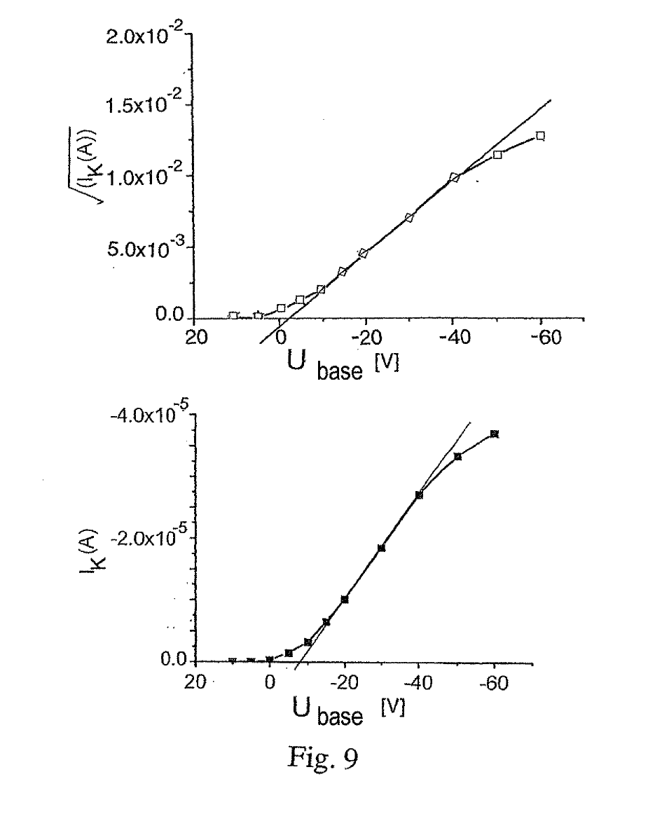

The result of the electrical characterization of this OFET (FIG. 9) is summarized in Tab. 1.

### Example 10

As an organic semiconductor, a solution of the compound (Z-4-a-1), of the compound (M-VI) and of the compound (P-I) in a weight ratio of 13:2:5 was prepared in a mixture of one part by volume of chloroform and one part by volume of toluene. Homogeneous films of visually good quality were obtained.

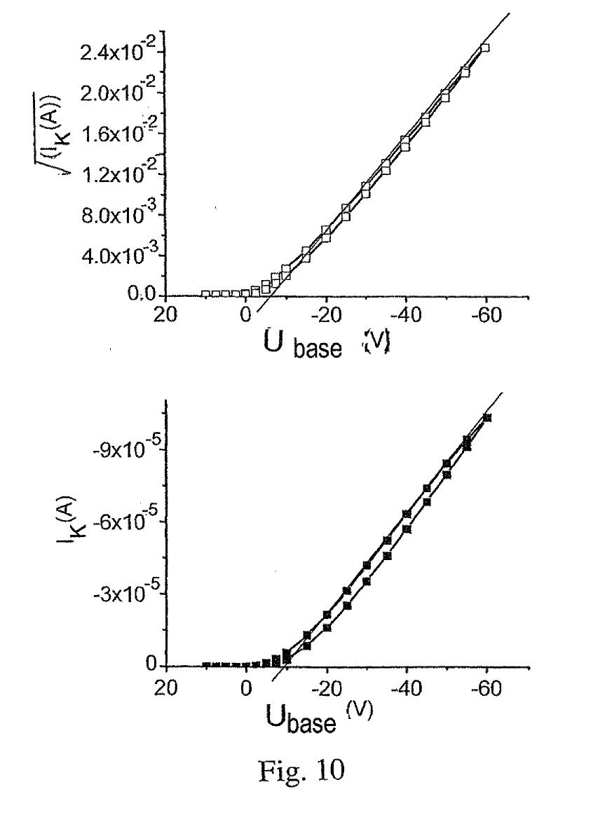

The result of the electrical characterization of this OFET (FIG. 10) is summarized in Tab. 1.

### Example 11

As an organic semiconductor, a solution of the compound of the formula (Z-4-a-3)

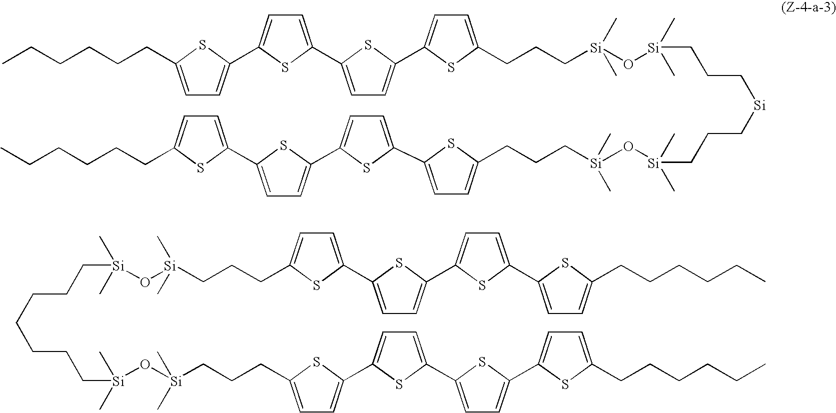

and of the compound (P-I) in a weight ratio of 1:1 was prepared in pure toluene. Homogeneous films of visually good quality were obtained.

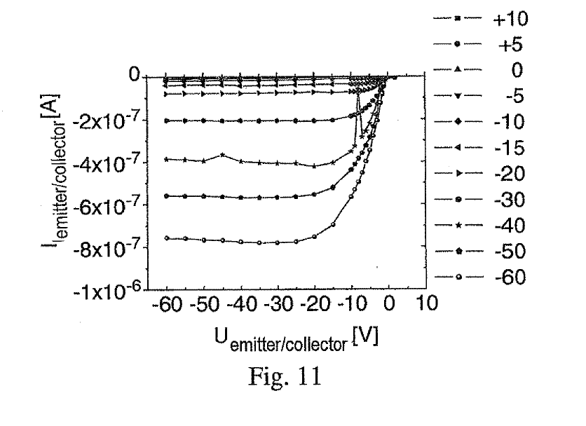

The result of the electrical characterization of this OFET (FIG. 11) is summarized in Tab. 1.

### Comparative Example 2

As an organic semiconductor, a solution of the compound (Z-4-a-1) in pure toluene was used. The resulting films exhibited visually irregular structures.

No electrical characteristic could be measured.

### Comparative Example 3

The preparation was effected as in Comparative Example 2, except that no organic dielectric intermediate layer was applied to the substrate. The resulting films exhibit visually irregular structures.

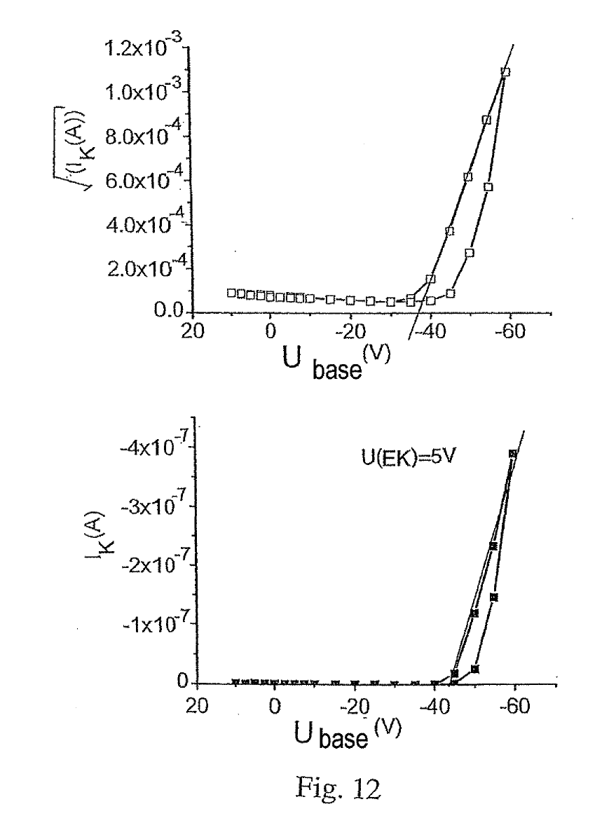

The result of the electrical characterization of this OFET (FIG. 12) is summarized in Tab. 1.

### Comparative Example 4

As an organic semiconductor, a solution of the compound (Z-4-a-3) in pure toluene was prepared. The resulting films exhibit visually irregular structures.

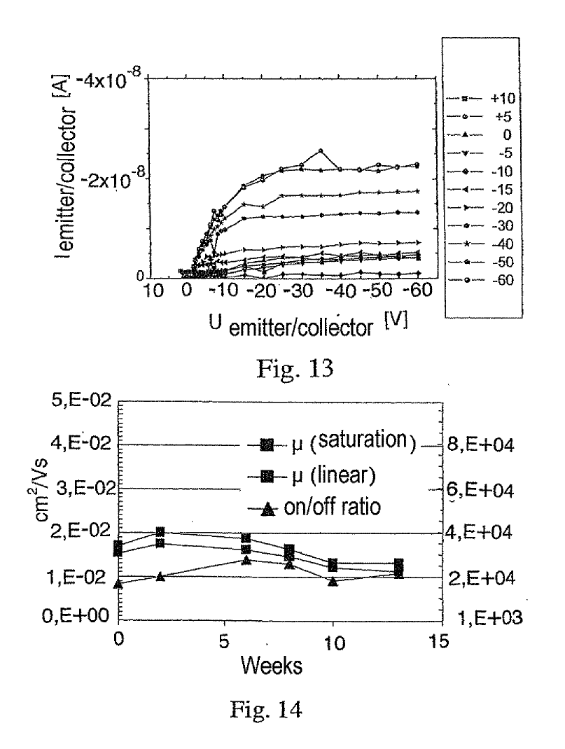

The result of the electrical characterization of this OFET (FIG. 13) is summarized in Tab. 1.

Table 1 summarizes the results of the transistor measurements.

TABLE 1

| | Mobility | Mobility | | Threshold |
|---|---|---|---|---|
| | (saturation) | (linear) | On/Off- | voltage |
| Example | [cm²/Vs] | [cm²/Vs] | ratio | [V] |
| 1 | 1.8E−2 | 1.8E−2 | 68800 | −8.4 |
| 2 | 2.7E−3 | 2.6E−3 | 3580 | −20.1 |
| 3 | 4.0E−4 | 2.9E−4 | 180 | −32.0 |
| 4 | 1.7E−2 | 1.5E−2 | 17400 | −20.6 |
| 5 | 7.6E−3 | 4.7E−3 | 23840 | −9.5 |
| 6 | 3.7E−2 | 4.3E−2 | 500 | −7.0 |
| 7 | 1.1E−2 | 1.2E−2 | 220 | −18.2 |
| 8 | 1.4E−4 | 1.0E−4 | 100 | −12.2 |
| 9 | 1.4E−2 | 1.9E−2 | 910 | −2.6 |
| 10 | 4.2E−2 | 4.6E−2 | 2460 | −5.1 |
| 11 | 3.9E−4 | 5.3E−4 | 100 | −20.0 |
| Comp. 1 | — | — | — | — |
| Comp. 2 | — | — | — | — |
| Comp. 3 | 2.6E−4 | 2.9E−4 | 100 | −36.9 |
| Comp. 4 | 8.8E−6 | 1.2E−5 | 10 | −25.1 |

1-26. (canceled)

27. An oligomeric compound of the general formula (M) 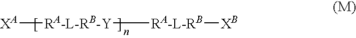
wherein L represents a linear conjugated oligomeric chain;
wherein each R^{A} and each R^{B} independently represents a moiety selected from the group consisting of linear or branched C₂-C₂₀-alkylene radicals, C₃-C₈-cycloalkylene radicals, mono- or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals;
wherein X^{A} and X^{B} each independently represents a moiety selected from optionally substituted vinyl groups, chlorine, iodine, hydroxyl, alkoxy groups having 1 to 3 carbon atoms, alkoxysilyl groups, silyl groups, chlorosilyl groups, siloxane groups, carboxyl groups, methyl or ethyl carbonate groups, aldehyde groups, methylcarbonyl groups, amino groups, amido groups, sulphone groups, sulphonic acid groups, halosulphonyl groups, sulphonate groups, phosphonic acid groups, phosphonate groups, trichloromethyl, tribromomethyl, cyanate groups, isocyanate groups, thiocyanate groups, isothiocyanate groups, cyano groups, nitro groups and H;
wherein each Y independently represents a moiety selected from an optionally substituted alkoxysilylene group, silylene group, chlorosilylene group, methylene group, dichloromethylene group, dibromomethylene group, divalent siloxane group, disiloxane group, carboxyl group, carbonate group, amino group, amido group, sulphate group, phosphonate group, phosphate group, borate group, S and O; and
wherein n represents an integer of 1 to 10.

28. The oligomeric compound according to claim 27, wherein L represents a linear conjugated oligomeric chain comprising one or more units selected from the group consisting of optionally substituted thiophene units, optionally substituted oligothiophene chains, oligo(3,4-ethylenedioxythiophene) chains having 2 to 8 optionally substituted thiophene or 3,4-ethylenedioxythiophene units, and combinations thereof.

29. The oligomeric compound according to claim 27, wherein each R^{A} and each R^{B} independently represent a linear or branched C₁-C₁₂-alkylene group and X^{A} and X^{B} each represent hydrogen.

30. The oligomeric compound according to claim 27, wherein at least one Y represents a divalent disiloxane group.

31. The oligomeric compound according to claim 27, wherein n is 1.

32. A mixture comprising at least one oligomeric compound according to claim 27 and at least one compound of the general formula (P) 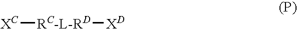
wherein L represent a linear conjugated oligomeric chain;
wherein R^{C} and R^{D} each independently represent a moiety selected from the group consisting of linear or branched C₂-C₂₀-alkylene radicals, C₃-C₈-cycloalkylene radicals, mono- or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals;
wherein X^{C} and X^{D} each independently represent a moiety selected from the group consisting of an optionally substituted vinyl group, a chlorine group, iodine group, hydroxyl group, alkoxy group having 1 to 3 carbon atoms, alkoxysilyl group, silyl group, chlorosilyl group, siloxane group, carboxyl group, methyl or ethyl carbonate group, aldehyde group, methyl-carbonyl group, amino group, amido group, sulphone group, sulphonic acid group, halosulphonyl group, sulphonate group, phosphonic acid group, phosphonate group, trichloromethyl group, tribromomethyl group, cyanate group, isocyanate group, thiocyanate group, isothiocyanate group, cyano group, nitro group and H.

33. The mixture according to claim 32, wherein L in the at least one compound of the general formula (P) represents a linear conjugated oligomeric chain comprising one or more units selected from the group consisting of optionally substituted thiophene units, optionally substituted oligothiophene chains, oligo(3,4-ethylenedioxythiophene) chains having 2 to 8 optionally substituted thiophene or 3,4-ethylenedioxythiophene units, and combinations thereof.

34. The mixture according to claim 32, wherein R^{C} and R^{D} each independently represent a linear or branched C₁-C₁₂-alkylene group and X^{C} and X^{D} are hydrogen.

35. The mixture according to claim 32, wherein L in general formula (M) and L in general formula (P) each represent the same linear conjugated oligomeric chain.

36. The mixture according to claim 32, wherein the at least one oligomeric compound and the at least one compound of the general formula (P) are present in a weight ratio of 20:80 to 80:20.

37. The mixture according to claim 32, further comprising at least one solvent selected from the group consisting of aromatics, ethers, halogenated aliphatic hydrocarbons and mixtures thereof.

38. A mixture comprising at least one oligomeric compound according to claim 27 and at least one macromolecular compound having a core-shell structure, wherein the core has a macromolecular base structure based on silicon and/or carbon and being bonded via a connecting chain based on carbon to at least three carbon-based linear oligomeric chains having conjugated double bonds throughout, and the linear conjugated chains each being saturated with at least one further chain.

39. The mixture according to claim 38, wherein the at least one macromolecular compound is of the general formula (Z):
K-[-V-L-R]_{q} (Z)
wherein K represents an n-functional core;
wherein each V represents a connecting chain;
wherein each L represents a linear conjugated oligomeric chain;
wherein each R represents a moiety selected from the group consisting of linear or branched C₂-C₂₀-alkyl radicals, C₃-C₈-cycloalkylene radicals, mono- or polyunsaturated C₂-C₂₀-alkenyl radicals, C₂-C₂₀-alkoxy radicals, C₂-C₂₀-aralkyl radicals, C₂-C₂₀-oligoether and C₂-C₂₀-polyether radicals; and
wherein q represents an integer greater than or equal to 3.

40. The mixture according to claim 38, wherein the core has a dendritic or hyperbranched structure.

41. The mixture according to claim 38, wherein the dendritic core comprises siloxane and/or carbosilane units.

42. The mixture according to claim 38, wherein at least one L in the shell represents a linear conjugated oligomeric chain comprising one or more units selected from the group consisting of optionally substituted thiophene units, optionally substituted oligothiophene chains, oligo(3,4-ethylenedioxythiophene) chains having 2 to 8 optionally substituted thiophene or 3,4-ethylenedioxythiophene units, and combinations thereof

43. The mixture according to claim 38, wherein the linear conjugated oligomeric chains of the macromolecular compound(s) are each saturated at their terminal linkage positions by identical or different, branched or unbranched alkyl or alkoxy groups.

44. The mixture according to claim 38, wherein the at least one oligomeric compound and the at least one macromolecular compound are present in a weight ratio of 10:90 to 90:10.

45. The mixture according to claim 38, wherein L in general formula (M) and at least one L in general formula (Z) each represent the same linear conjugated oligomeric chain.

46. The mixture according to claim 38, further comprising at least one solvent selected from the group consisting of aromatics, ethers, halogenated aliphatic hydrocarbons and mixtures thereof.

47. The mixture according to claim 38, further comprising at least one compound of the general formula (P) 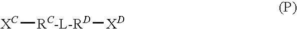
wherein L represent a linear conjugated oligomeric chain;
wherein R^{C} and R^{D} each independently represent a moiety selected from the group consisting of linear or branched C₂-C₂₀-alkylene radicals, C₃-C₈-cycloalkylene radicals, mono- or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals;
wherein X^{C} and X^{D} each independently represent a moiety selected from the group consisting of an optionally substituted vinyl group, a chlorine group, iodine group, hydroxyl group, alkoxy group having 1 to 3 carbon atoms, alkoxysilyl group, silyl group, chlorosilyl group, siloxane group, carboxyl group, methyl or ethyl carbonate group, aldehyde group, methyl-carbonyl group, amino group, amido group, sulphone group, sulphonic acid group, halosulphonyl group, sulphonate group, phosphonic acid group, phosphonate group, trichloromethyl group, tribromomethyl group, cyanate group, isocyanate group, thiocyanate group, isothiocyanate group, cyano group, nitro group and H.

48. A mixture comprising:
(i) at least one compound of the general formula (P) 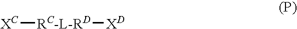
wherein L represent a linear conjugated oligomeric chain;
wherein R^{C} and R^{D} each independently represent a moiety selected from the group consisting of linear or branched C₂-C₂₀-alkylene radicals, C₃-C₈-cycloalkylene radicals, mono- or polyunsaturated C₂-C₂₀-alkenylene radicals, C₂-C₂₀-oxyalkylene radicals, C₂-C₂₀-aralkylene radicals or C₂-C₂₀-oligo- or C₂-C₂₀-polyether radicals;
wherein X^{C} and X^{D} each independently represent a moiety selected from the group consisting of an optionally substituted vinyl group, a chlorine group, iodine group, hydroxyl group, alkoxy group having 1 to 3 carbon atoms, alkoxysilyl group, silyl group, chlorosilyl group, siloxane group, carboxyl group, methyl or ethyl carbonate group, aldehyde group, methyl-carbonyl group, amino group, amido group, sulphone group, sulphonic acid group, halosulphonyl group, sulphonate group, phosphonic acid group, phosphonate group, trichloromethyl group, tribromomethyl group, cyanate group, isocyanate group, thiocyanate group, isothiocyanate group, cyano group, nitro group and H; and
(ii) at least one macromolecular compound with core-shell structure, the core having a macromolecular base structure based on silicon and/or carbon and being bonded via a connecting chain based on carbon to at least three carbon-based linear oligomeric chains having conjugated double bonds throughout, and the linear conjugated chains each being saturated with at least one further chain.

49. An electronic component comprising an oligomeric compound according to claim 1.
